(19) United States
(12) Patent Application Publication
(10) Pub. No.: US 2010/0294852 A1
(43) Pub. Date: Nov. 25, 2010

(54) Methods of Dispensing Two or More Volatile Materials

(76) Inventors: Michael J. Banco, Racine, WI (US); René Maurice Béland, Waterford, WI (US); Mark Groth, Racine, WI (US); Scott J. Landy, Grayslake, IL (US); Amelia H. Majerowski, Kenosha, WI (US)

Correspondence Address: S.C. JOHNSON & SON, INC., 1525 HOWE STREET, RACINE, WI, 53403-2236, US

(21) Appl. No.: 12/785,238

(22) Filed: May 21, 2010

Related U.S. Application Data
(60) Provisional application No. 61/216,796, filed on May 21, 2009.
(60) Provisional application No. 61/235,918, filed on Aug. 21, 2009.

(51) Int. Cl. A61L 9/04

(52) U.S. Cl. 239/6

(57) ABSTRACT

A method of dispensing two or more volatile materials includes the step of providing a volatile material dispenser having at least one diffusion element and adapted to accommodate two or more containers having volatile materials therein, wherein each of the volatile materials is subjected to a diffusion element. The method further includes the steps of emitting a first of the volatile materials at a first intensity level for a first period of time between about 30 minutes and about 2 hours and emitting a second of the volatile materials at a second intensity level for a second period of time following the first period of time. The second period of time is between about 5 minutes and about 30 minutes and the second intensity level is greater than the first intensity level. The method still further includes the step of repeating the step of emitting the first volatile material.

### CROSS REFERENCE TO RELATED APPLICATIONS

This application claims the benefit of U.S. Provisional Application Ser. No. 61/216,796, filed May 21, 2009, and entitled “Method and Apparatus for Dispensing Fragrances,” and U.S. Provisional Application Ser. No. 61/235,918, filed Aug. 21, 2009, and entitled “Method and Apparatus for Dispensing Fragrances,” the disclosures of which are incorporated herein in their entireties.

### REFERENCE REGARDING FEDERALLY SPONSORED RESEARCH OR DEVELOPMENT

Not applicable

### SEQUENTIAL LISTING

Not applicable

### FIELD OF THE INVENTION

The present invention generally relates to a volatile material dispensing system, more particularly, to a volatile material dispensing system and method of dispensing volatile materials from same that increase user perception of the volatile materials.

### BACKGROUND OF THE INVENTION

It is known that a user's perception of a dispensed fragrance of a constant intensity tends to decay over time. This decay in perception, which is commonly referred to as adaptation and/or habituation, reduces the user's enjoyment of the dispensed fragrance. Adaptation and/or habituation are the reduction of physiological, psychological, or behavioral response occurring when a specific stimulus occurs repeatedly. It is generally believed that adaptation and/or habituation with respect to a fragrance can be reduced by changing the level of intensity of the dispensed fragrance or by dispensing a different fragrance. Fragrance dispensers and methods of dispensing fragrances that address the issue of adaptation and/or habituation are known in the art.

One such fragrance dispenser emits a first fragrance for a first period of time followed by emission of a second fragrance for a second period of time followed by emission of a third fragrance for a third period of time. A further pattern or algorithm for dispensing fragrances includes emission of a first fragrance in repeated short intermittent bursts during a first period of time, the emission of a second fragrance in repeated short intermittent bursts during a second period of time, and the emission of a third fragrance in repeated short intermittent bursts during a third period of time.

Another dispenser emits fragrances in an alternating sequence while the dispenser is activated. The dispenser includes, for example, first and second heaters for emitting first and second fragrances, respectively. In one embodiment, the fragrances are alternatively emitted by deactivating one of the heaters at the same time the other of the heaters is activated. Alternatively, one of the heaters may be deactivated followed by a gap period and then the other of the heaters may be activated. Still further, one of the heaters may be activated before the other of the heaters is deactivated to create an overlap period.

Existing devices offering solutions to adaptation and/or habituation may change dispensed fragrances or intensities thereof frequently over a period of a day or several hours, for example, every 45 minutes, thereby exposing a user to a seemingly constant change of fragrance.

### SUMMARY OF THE INVENTION

In a first embodiment of the present invention, a method of dispensing two or more volatile materials includes the step of providing a volatile material dispenser having at least one diffusion element and adapted to accommodate two or more containers having volatile materials therein, wherein each of the volatile materials is subjected to a diffusion element. The method further includes the steps of emitting a first of the volatile materials at a first intensity level for a first period of time between about 30 minutes and about 2 hours and emitting a second of the volatile materials at a second intensity level for a second period of time following the first period of time. The second period of time is between about 5 minutes and about 30 minutes and the second intensity level is greater than the first intensity level. The method still further includes the step of repeating the step of emitting the first volatile material.

In a second embodiment of the present invention, a method of dispensing two or more volatile materials includes the step of providing a volatile material dispenser having two or more diffusion elements and adapted to accommodate two or more containers having volatile materials therein, wherein each of the volatile materials is subjected to one of the diffusion elements. The method further includes the steps of emitting a first of the volatile materials for a first period of time and emitting a second of the volatile materials for a second period of time following the first period of time. Still further, the method includes the step of refraining from emission of any volatile material for a third period of time following the second period of time. The first, second, and third periods of time make up an emission cycle that is repeated, wherein the emission cycle is greater than or equal to 24 hours.

In a further embodiment of the present invention, a method of dispensing two or more volatile materials includes the step of providing a volatile material dispenser having at least one diffusion element and adapted to accommodate two or more containers having volatile materials therein, wherein each of the volatile materials is subjected to a diffusion element. The method further includes the step of emitting a first volatile material from a first of the containers for a first generally continuous period of time, wherein after the first period of time, the first volatile material in the first container is substantially depleted. Still further, the method includes the step of emitting a second volatile material from a second of the containers for a second generally continuous period of time, wherein after the second period of time, the second volatile material in the second container is substantially depleted.

### BRIEF DESCRIPTION OF THE DRAWINGS

FIG. 1 is a front elevational view of an apparatus for dispensing up to three volatile materials;

FIG. 2 is a side elevational view of the apparatus of FIG. 1;

FIG. 3 illustrates a scented oil container for use with the apparatus of FIGS. 1 and 2;

FIG. 4 is a front elevational view of another apparatus for dispensing up to three volatile materials;

FIG. 5 illustrates a fragrance laden gel refill cartridge for use with the apparatus of FIG. 4;

FIG. 6 is a front elevational view of a cover for the apparatus of FIG. 4;

FIG. 7 is a cross-sectional view of a base, the refill cartridge, and the cover of FIGS. 4-6, respectively, in an assembled state, taken generally along the lines 7-7 of FIG. 4;

FIG. 8 is an exploded view of the assembled apparatus and gel refill cartridge of FIGS. 4-7;

FIGS. 9A-9D are diagrammatic representations of various examples of a first mode of dispensing for implementation by any of the dispensing apparatuses disclosed herein;

FIG. 10A is a diagrammatic representation of a second mode of dispensing for implementation by any of the dispensing apparatuses disclosed herein;

FIG. 10B is a diagram representing a sample execution of an implementation of the second mode of dispensing;

FIG. 11 is a graph of data for perceived fragrance intensities over time;

FIGS. 12A and 12B are diagrammatic representations of examples of a third mode of dispensing for implementation by any of the dispensing apparatuses disclosed herein;

FIGS. 13A and 13B are diagrammatic representations of examples of a fourth mode of dispensing for implementation by any of the dispensing apparatuses disclosed herein;

FIG. 14A is a graph of data for rates of weight loss over time for a first set of fragrances dispensed by a dual fragrance dispenser having heaters and a fan;

FIG. 14B is another graph of data for rates of weight loss over time for a second set of fragrances dispensed by a dual fragrance dispenser having heaters and a fan;

FIG. 14C is a graph of data for rates of weight loss over time for a third set of fragrances dispensed by a single fragrance dispenser having a heater and a fan;

FIG. 14D is a graph of data for rates of weight loss over time for fragrances dispensed by a single fragrance dispenser having only a heater;

FIG. 14E is a graph of the data in the graphs of FIGS. 13A-13D, wherein each set of data has been normalized;

FIG. 14F is a graph of the averaged normalized data of FIG. 13E;

FIG. 15A is a diagrammatic representation of a fifth mode of dispensing;

FIG. 15B is another diagrammatic representation of the fifth mode of dispensing;

FIG. 15C is a further diagrammatic representation of the fifth mode of dispensing;

FIG. 16 is yet another diagrammatic representation of the fifth mode of dispensing;

FIGS. 17 and 18 are graphs representing responses from respondents in first and second study implementations of two embodiments of the fifth mode of dispensing;

FIG. 19A is a front elevational view of an apparatus for dispensing up to four volatile materials; and

FIG. 19B is a diagram representing a sample execution of an implementation of the fifth mode of dispensing.

Other aspects and advantages of the present invention will become apparent upon consideration of the following detailed description, wherein similar structures have similar reference numbers.

### DETAILED DESCRIPTION

The present invention is directed to an apparatus and method for dispensing volatile materials that increase a user's perception of the dispensed volatile materials. While specific embodiments are discussed herein, it is understood that the present invention is to be considered only as an exemplification of the principles of the present invention. For example, lengths of time periods as disclosed for each of the modes of dispensing hereinbelow are approximate and may not be absolute but rather may be intended to impart the relative lengths of the time periods with respect to one another. Therefore, the present invention is not intended to limit the invention to the embodiments illustrated.

Further, although the specific embodiments herein refer to fragrances rather than volatile materials, it is to be understood that any type of volatile material emitting an aroma or scent may be utilized with any of the apparatuses and modes of dispensing as disclosed herein. Illustratively, the types of volatile materials may be, for example, a fragrance, an aromatherapy scent, a positive fragrancing active material, an air-freshener, or the like, and combinations thereof.

It is desirable to produce a volatile material dispenser that operates to dispense volatile materials as aerosols, scented oils, or gels, for example, and may be enhanced with the addition of a diffusion element, such as for example, a heater, a fan, a piezoelectric device, an aerosol dispenser, and/or any other known volatile dispenser. It is desirable that the volatile material dispenser produces a long lasting pleasurable aroma while inhibiting the onset of adaptation and/or habituation to the aroma. Prior approaches to address the onset of adaptation and/or habituation include modes of dispensing that include alternation or variation of one or more volatile materials over time, variation of the level of intensity of one or more volatile materials over time, and the variation of time of dispensing of one or more volatile materials.

Those having skill in the art of fragrance dispensers recognize that the alternation of volatile materials, in particular fragrances, is most effective at minimizing adaptation and/or habituation when emission periods for each of the fragrances are shorter in length, such as an hour or less. It is also recognized that the longer the emission periods for each of the fragrances, the more likely adaptation and/or habituation are to set in. In particular, one skilled in the art would expect a mode of dispensing having emission periods for each fragrance of six hours to promote the onset of adaptation and/or habituation. However, as discussed in detail hereinbelow, test results indicate that a mode of dispensing having alternating emission periods of about six hours is more effective in inhibiting adaptation and/or habituation than a mode of dispensing having alternating emission periods of about 45 minutes.

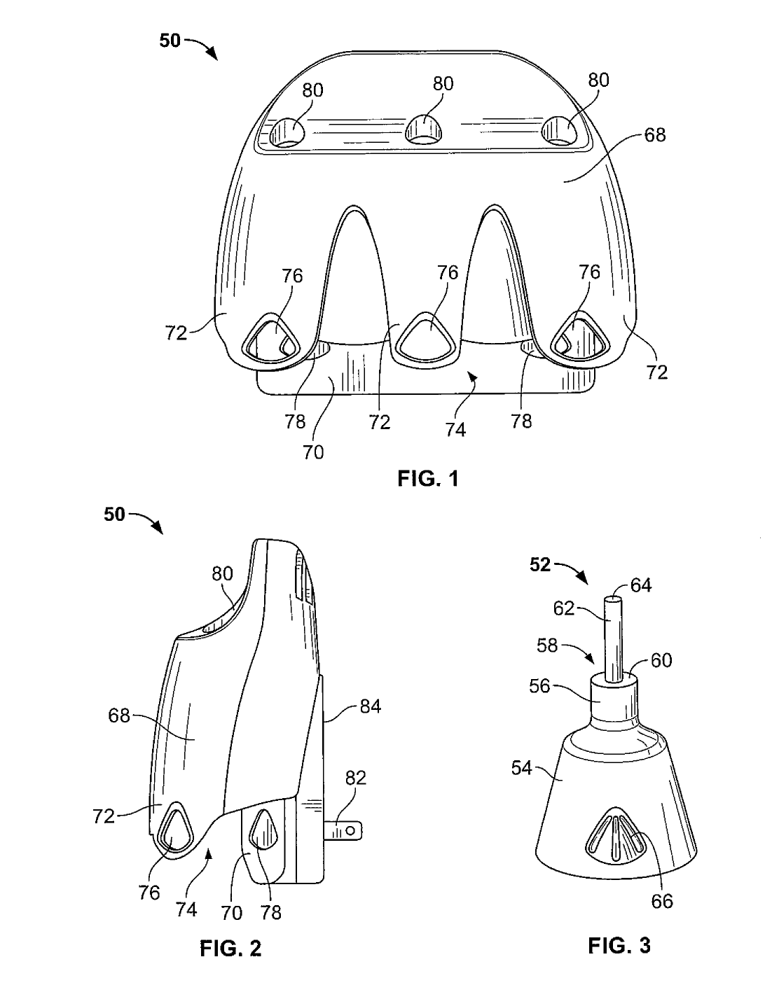

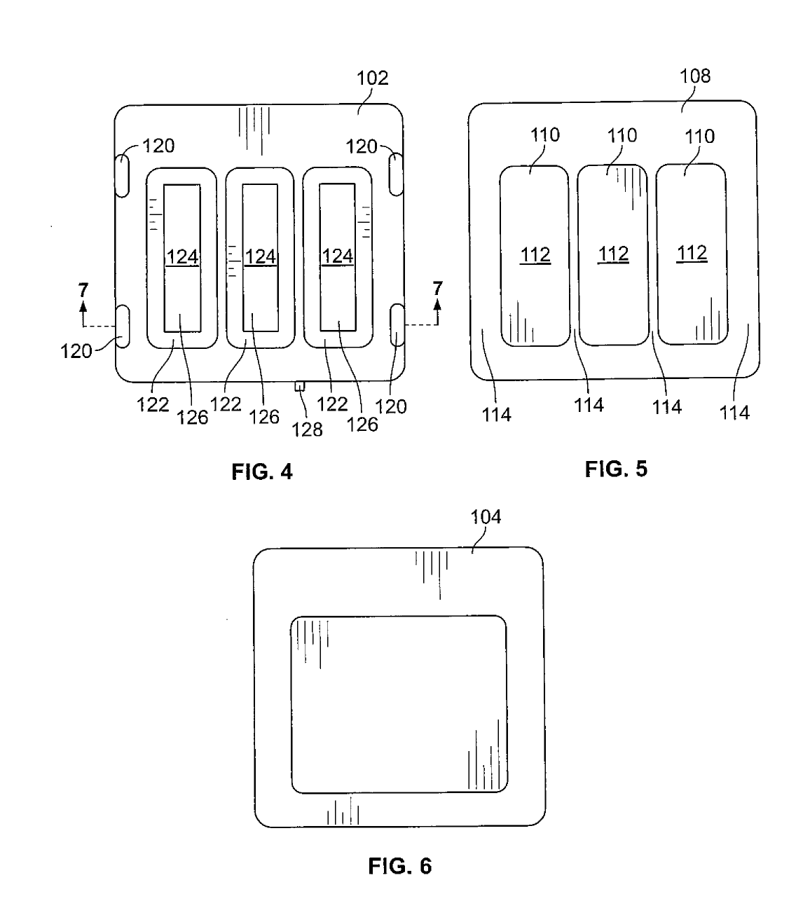

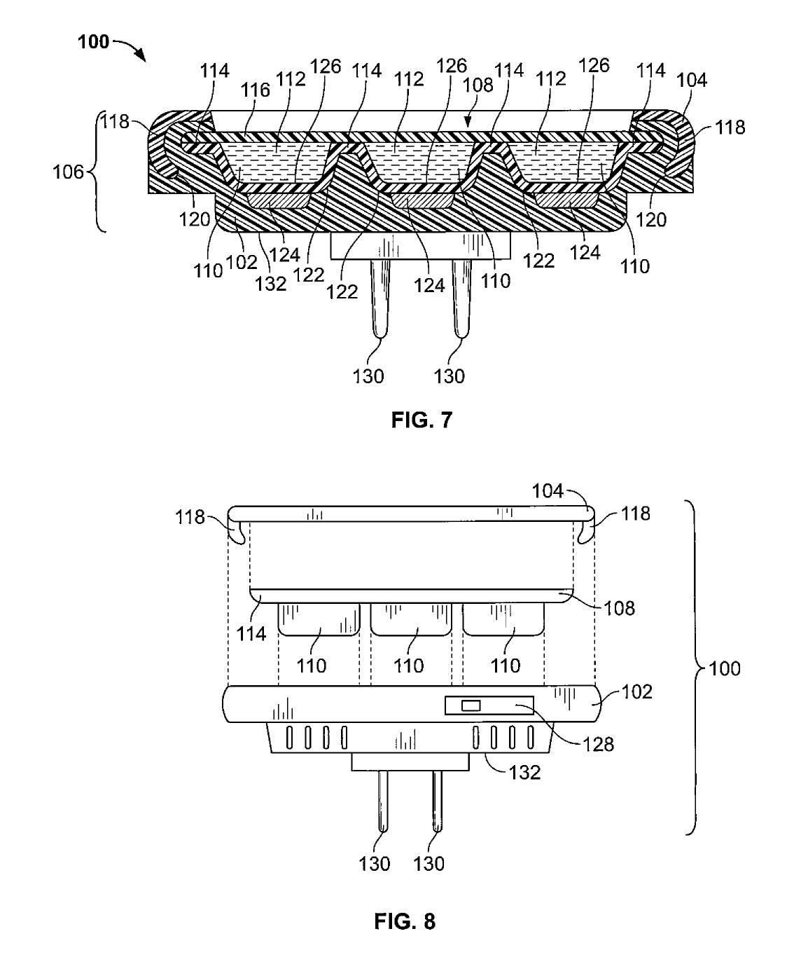

Turning now to the figures, an illustrative volatile material dispenser 50 is shown in FIGS. 1 and 2. The dispenser 50 is designed to accommodate up to three scented oil fragrance containers, for example, the container 52 illustrated in FIG. 3. The container 52 includes a body 54 having a scented oil fragrance disposed therein. The container 52 further includes a neck 56 and an aperture 58 disposed in a distal end 60 of the neck 56. A wick 62 is held by the neck 56 such that a first end (not visible) of the wick 62 is disposed in the body 54 of the container 52 in contact with the fragrance and a second end 64 of the wick 62 extends upwardly from the neck 56 out of the container 52.

Protrusions 66, for example, fan-shaped protrusions as shown in FIG. 3, extend from front and back surfaces of the body 54 of the container 52. As seen in FIGS. 1 and 2, the dispenser 50 includes a housing 68 having an interior wall 70 and three skirts 72 that extend around and define corresponding cavities 74 between the interior wall 70 and the skirts 72. Each of the skirts 72 includes an aperture 76 disposed therethrough and configured to accept a corresponding protrusion 66 that extends from the body 54 of the container 52. Shell-shaped indentations 78 are disposed in the interior wall 70 of the housing 68 opposite each of the apertures 76. When the container 52 is inserted into one of the cavities 74, the protrusions 66 are captured within the corresponding aperture 76 and the corresponding indentation 78 to hold the container 52 in place within the housing 68.

When the containers 52 are inserted into the housing 68, the wick 62 of each container 52 extends upwardly through corresponding channels 80 within the dispenser housing 68 and in proximity to corresponding heating elements (not shown) such that the second end 64 of each wick 62 may be visible through a top end of each channel 80. Each heating element is independently controllable by a control circuit or controller (not shown) disposed within the housing 68. As more fully discussed hereinbelow, the controller controls the amount and temporal distribution of power to each of the heating elements. Electrical blades or prongs 82 extend from a rear surface 84 of the dispenser 50, wherein the prongs 82 may be inserted into a convention electrical socket to allow access to a source of external power.

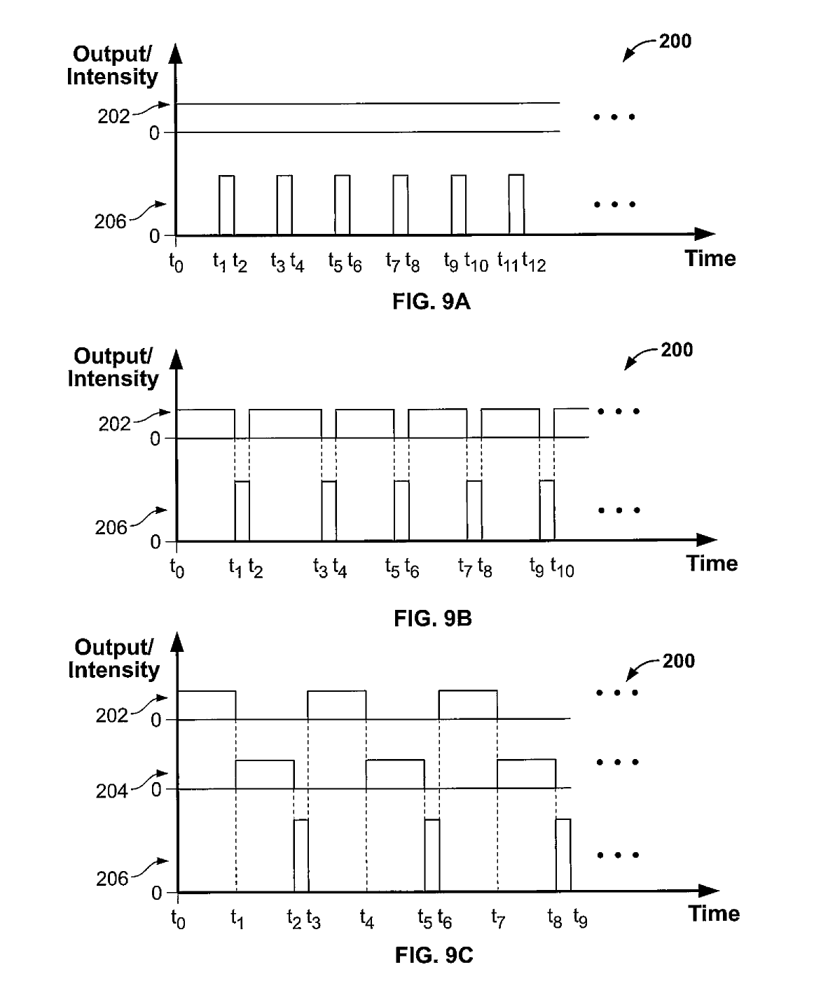

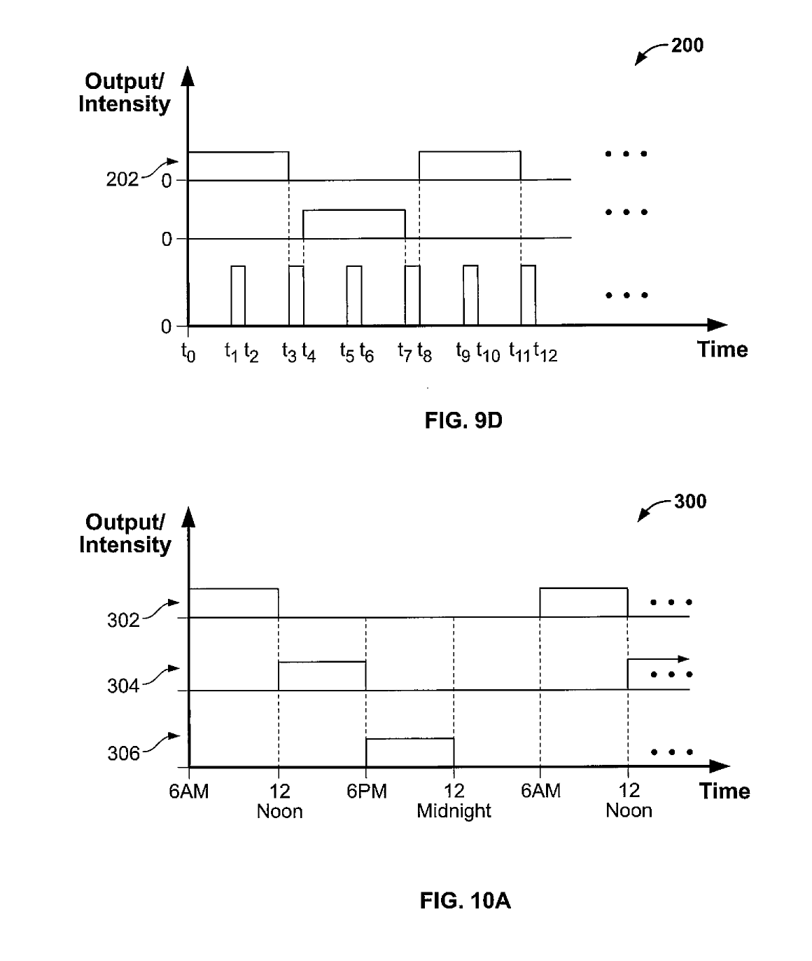

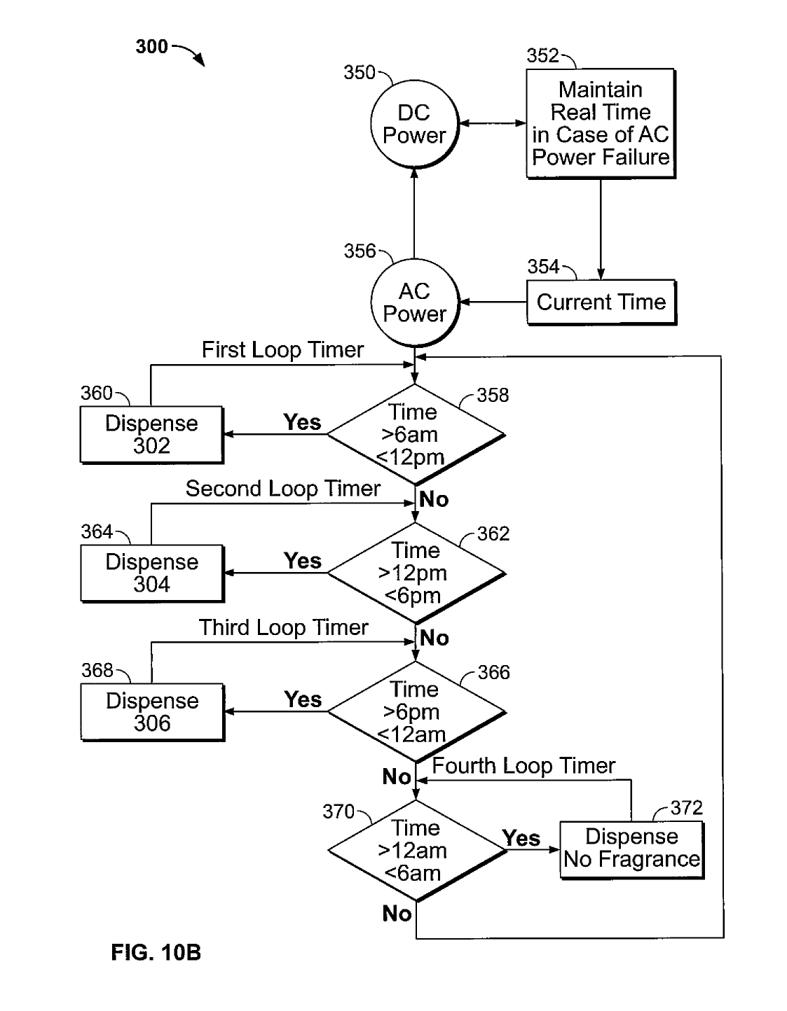

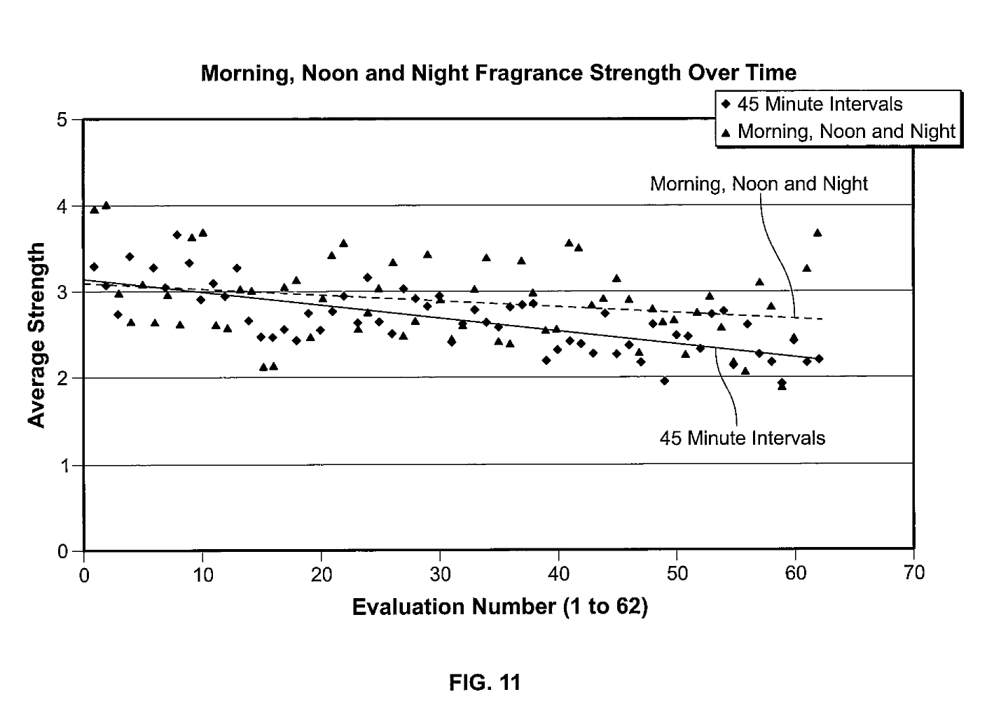

Referring to FIGS. 4-8, a further volatile material dispenser 100 includes a base 102 and a cover 104 that together comprise a housing 106, as illustrated in FIG. 7. The housing 106 is designed to accommodate a volatile material container 108 including a plurality of independent reservoirs 110, for example, three, that each hold a volatile material 112, for example, a fragrance laden gel therein. Each of the plurality of independent reservoirs 110 is entirely surrounded by a flange 114. A non-porous permeable membrane 116 (see FIG. 7) is adhered to the flange 114 to cover each of the plurality of reservoirs 110 and extends across the container 108. The cover 104 releasably attaches to the base 102, for example, by a frictional fit. The releasable attachment may be more of a snap fit such that teeth 118 snap into recesses 120.

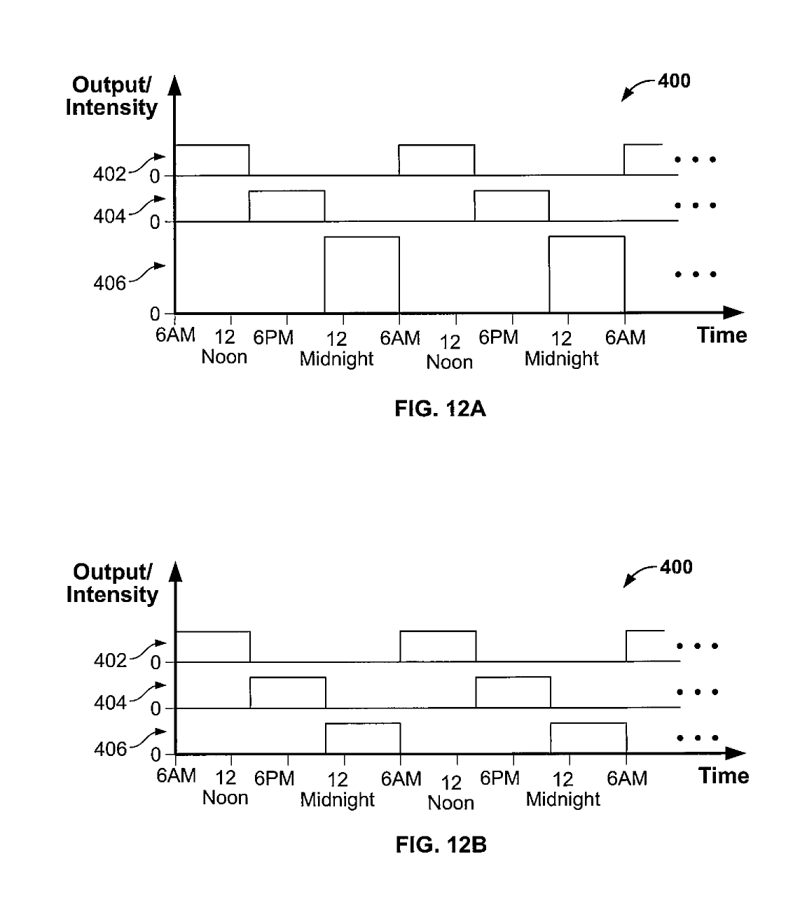

Referring to FIGS. 4 and 7, the base 102 includes a plurality of heating pans 122. A heating element 124 is centrally disposed under each heating pan 122 such that an exposed surface 126 of the heating element 124 is approximately flush with a surrounding surface of the corresponding heating pan 122. Each heating element 124 is independently controllable by a control circuit or controller (not shown) disposed within the base 102. Referring to FIGS. 4 and 8, the base 102 may also include a mode selector switch 128, for example, in electrical communication with the controller. As more fully discussed hereinbelow, the controller in conjunction with the mode selector switch 128, controls the amount and temporal distribution of power to each of the heating elements 124. Referring to FIGS. 7 and 8, electrical blades or prongs 130 extend from a rear surface 132 of the base portion 102, wherein the prongs 130 may be inserted into a convention electrical socket to allow access to a source of external power.

The volatile material container 108 may be similar to the volatile material holders described in Christianson, Air Freshener with Picture Frame, U.S. Pat. No. 7,441,360, which is herein incorporated by reference in its entirety. Other illustrative volatile material dispensers useful for implementing the modes of operation disclosed herein include those disclosed in, for example, Schramm et al., Apparatus for and Method of Dispensing Active Materials, U.S. patent application Ser. No. 11/427,714; Neumann et al., Fragrance Dispenser, U.S. patent application Ser. No. 12/319,606; and Porchia et al., Volatile Material Diffuser, U.S. patent application Ser. No. 12/288,606, the disclosures of which are hereby incorporated herein by reference in their entireties. Further, any device for emitting two or more volatile materials may be utilized to implement the modes of dispensing disclosed hereinbelow.

Several modes of dispensing a fragrance while inhibiting adaptation and/or habituation with regard to the fragrance are included in this disclosure. Any of the modes of dispensing presented herein may be implemented utilizing any of the apparatuses presented herein or any other apparatus employing a proper number of diffusion elements and containers.

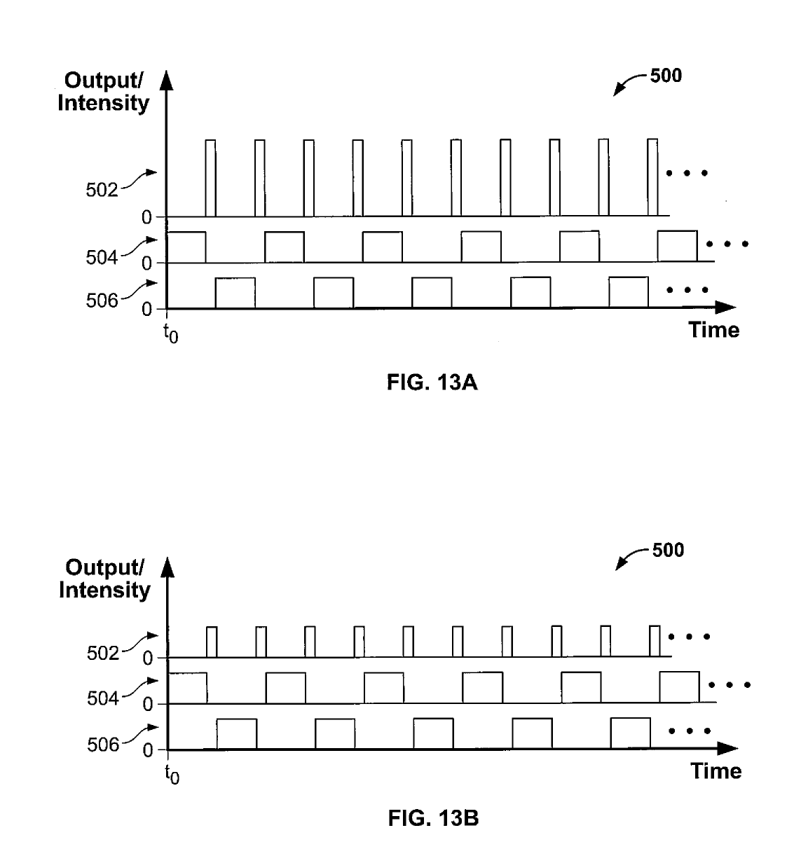

In a first mode 200 of dispensing, as seen in FIGS. 9A-9D, two or more base fragrances are dispensed having unequal intensities and for unequal emission periods. In one illustrative example implementing the first mode 200 of dispensing and referring to FIG. 9A, a first base fragrance 202 is continuously dispensed with a weaker intensity for a longer emission period than a second, burst fragrance 206, which is conversely dispensed with a stronger intensity for a shorter emission period than the first base fragrance 202. The burst fragrance 206 is not emitted and emitted for alternating dwell and burst periods. The burst fragrances 206 disclosed herein are interrupting scents that are distinctive and intended to be recognizable to a user. In essence, an interrupting scent provides a distinct barrier between base fragrances such that a user may experience distinct base fragrances rather than a blending of base fragrances. If the interrupting scent is provided during a continuous single base fragrance, the interrupting scent reawakens the user's senses such that they begin to smell the base fragrance again. The interrupting scent may be, for example, a clean scent such as bleach, citrus, or pine, a fresh scent such as rosemary, mint, spearmint chocolate, or eucalyptus, an invigorating scent such as coffee or candy cane, a scent having high hedonic characteristics such as apple, combinations thereof, and the like.

The dwell periods are depicted in FIG. 9A as between t₀ and t₁, t₂ and t₃, t₄ and t₅, etc. Likewise, the burst periods are shown as emission periods t₁ to t₂, t₃ to t₄, t₅ to t₆, etc. The dwell periods are preferably between about 30 minutes and about 2 hours in length, more preferably between about 45 minutes and about 1 hour in length and most preferably about 45 minutes in length and the burst periods are preferably between about 1 minute and about 30 minutes in length, more preferably between about 5 minutes and about 20 minutes in length, and most preferably about 15 minutes in length. Optionally, the example of FIG. 9A may be utilized with multiple base fragrances to create a continuous blending of fragrances, wherein the burst fragrance 206 intermittently cleans the air of the blended base fragrances.

FIG. 9B illustrates a second example of an implementation of the first mode 200 of dispensing that is similar to the implementation of FIG. 9A. The first base fragrance 202 is dispensed for emission periods of between about 30 minutes and about 2 hours in length, more preferably between about 45 minutes and about 1 hour, and most preferably about 45 minutes, as seen between times t₀ and t₁, t₂ and t₃, t₄ and t₅, t₆ and t₇, etc. During the periods that the first base fragrance 202 is not emitted, such as between times t₁ and t₂, t₃ and t₄, t₅ and t₆, etc., the burst fragrance 206 is emitted for emission periods of between about 1 minute and about 30 minutes, more preferably between about 5 minutes and about 20 minutes, and most preferably about 15 minutes.

Referring to FIG. 9C, a further illustrative implementation of the first mode 200 of dispensing includes first and second base fragrances 202, 204, dispensed with weaker intensities for longer emission periods than a third, burst fragrance 206, which is conversely dispensed with a stronger intensity but for a shorter emission period than the first and second base fragrances 202, 204. The first and second base fragrances 202, 204 are emitted for alternating emission periods during the time periods t₀ to t₁ and t₁ to t₂, respectively. Thereafter, the burst fragrance 206 is emitted at a stronger intensity, as seen between times t₂ and t₃. As seen in FIG. 9C, this pattern is repeated until the device implementing such mode of dispensing is deactivated. Optionally, rather than alternating the first and second base fragrances 202, 204, the first and second base fragrances 202, 204 may be emitted at the same time to create a blending of fragrances followed by a time period of no emission of the first and second base fragrances 202, 204 and emission of the burst fragrance 206. The emission period for each of the first and second base fragrances is between about 30 minutes and about 2 hours, more preferably between about 45 minutes and about 1 hour, and most preferably about 45 minutes. If more than two base fragrances are utilized, similar emission periods are used. Further, the emission periods of the burst fragrance 206 are the same as with respect to FIGS. 9A and 9B.

In yet another illustrative example implementing the first mode 200 of dispensing, as seen in FIG. 9D, the first and second base fragrances 202, 204 are emitted with weaker intensities in an alternating fashion for emission periods with gap periods therebetween. The burst fragrance 206 is emitted at a stronger intensity for a shorter period of time during emission of the first and second base fragrances 202, 204 as defined between times t₁ and t₂, t₅ and t₆, etc. and during the gap periods as defined between times t₃ and t₄, t₇ and t₈, etc. The emission periods of each of the first and second base fragrances 202, 204 and the emission periods of the burst fragrance 206 are the same as with respect to FIG. 9C. If the first and second base fragrances 202, 204 are emitted for longer emission periods of time, multiple emissions of the burst fragrance 206 may be utilized during such base fragrance emission period.

Referring to the first mode 200 of FIGS. 9A-9D, if an aerosol or intermittent dispenser is utilized, similar emission periods as described with respect to FIGS. 9A-9D for the first and second fragrances 202, 204 and burst fragrance 206 are utilized, except that during the emission periods, the respective fragrances 202, 204, 206 are intermittently emitted.

In a second mode 300 of dispensing, two or more fragrances are dispensed during an emission cycle, which is repeated. An emission cycle is defined as dispensing of each of the fragrances once (with or without gaps or overlaps between fragrances). An emission cycle is preferably greater than or equal to about 24 hours, more preferably between about 24 hours and 7 days, and most preferably about 24 hours. After the emission cycle is over, a further emission cycle begins and this pattern repeats until the second mode 300 of dispensing is interrupted (or the device in which it is implemented is deactivated). During an emission cycle, any number of fragrances may be utilized with gaps and/or overlaps therebetween.

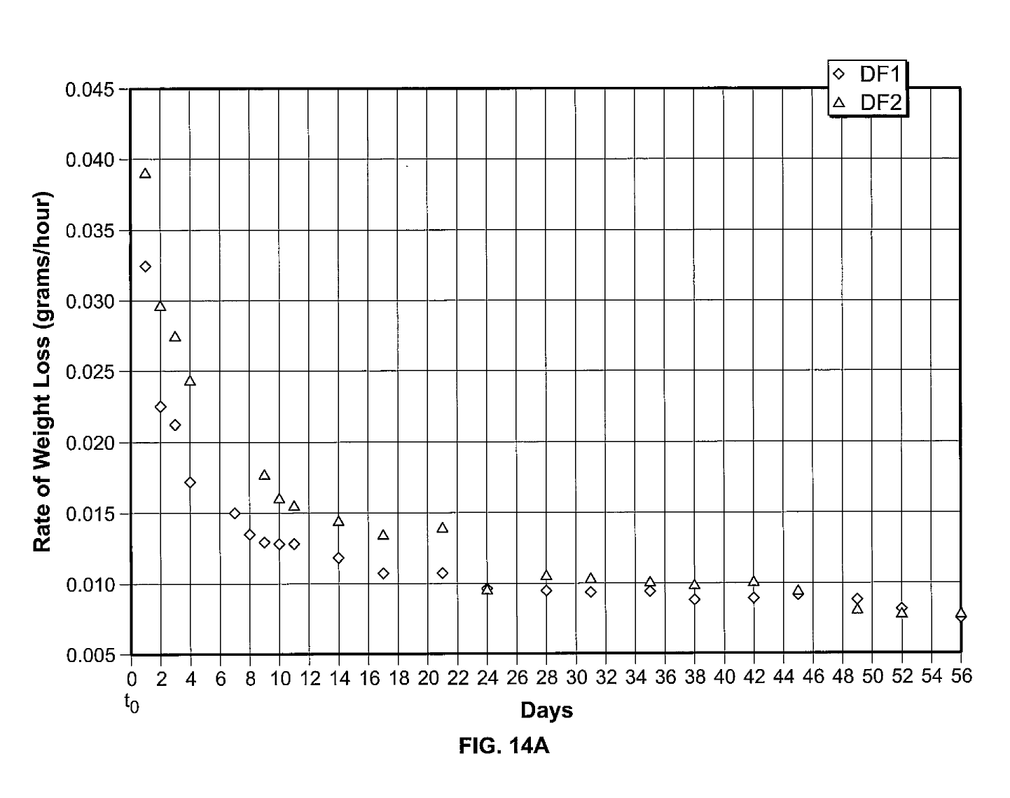

In a first example of the second mode 300, the emission cycle is 24 hours or 1 day. During such emission cycle, three fragrances may be emitted that correlate to different times of the day and each fragrance further meets the needs of a user for that period of time during the day. For example, during the morning hours, a user may desire an awakening, rejuvenating, and/or revitalizing scent, during the afternoon hours, a user may desire an invigorating, energetic, and/or refreshing scent, and during the evening hours, a user may desire a relaxing, refreshing, and/or sleep-inducing scent. In a specific example and referring to FIG. 10A, an emission cycle of 24 hours is utilized and a first fragrance 302 that is perceived by a user as energizing is dispensed during the morning hours of the day, for example, between 6 a.m. and 12 noon. A second fragrance 304 that is perceived by the user to allow for maximum productivity or motivation is dispensed during the afternoon hours of the day, for example, between 12 noon and 6 p.m., and a third fragrance 306 that is perceived by a user as relaxing is delivered during the evening hours of the day, for example, between 6 p.m. and 12 midnight. Between the evening hours and the morning hours, for example between 12 midnight and 6 a.m., dispensing of fragrance may be diminished or cease entirely for a dwell period to save on the amount of fragrance dispensed and to allow the user's olfactory system to recover from prior fragrance stimuli to further overcome and inhibit adaptation and/or habituation.

In a further example of the second mode 300 utilizing an emission cycle of 24 hours and dispensing three fragrances, each of the three fragrances is emitted for a period of 7 hours, for example, a first fragrance is emitted between 4 a.m. and 11 a.m., a second fragrance is emitted between 11 a.m. and 6 p.m., and a third fragrance is emitted between 6 p.m. and 1 a.m. Emission of the three fragrances would thereafter be followed by a 3 hour dwell period in which no fragrance is emitted and the emission cycle, including emission of all three fragrances and the dwell period, would be repeated beginning at 4 a.m. In yet another embodiment of the second mode 300 in which an emission cycle of 24 hours and two fragrances are utilized, each of the two fragrances is emitted for a period of 10 hours, for example, a first fragrance is emitted between 4 a.m. and 2 p.m and a second fragrance is emitted between 2 p.m. and 12 a.m. A dwell period of 4 hours would then follow emission of the two fragrances with a subsequent emission cycle, including emission of both fragrances and the dwell period, beginning at 4 a.m.

Employing any emission cycle greater than or equal to about 24 hours, two or more fragrances may each be emitted in the second mode 300 for emission periods constituting between about 10% and about 50% of the emission cycle, more preferably between about 20% and about 40% of the emission cycle, and more preferably about 30% of the emission cycle. The fragrances may also be emitted with a period of low or no fragrance emission that constitutes between about 1% and about 30% of the emission cycle, more preferably between about 10% and about 20% of the emission cycle, and most preferably between about 12% and about 17% of the emission cycle.

Although specific times are utilized in the foregoing examples of the second mode 300, such times may be altered based on a preset program or based on user preference. Further, although the examples herein include fragrances that are emitted for similar emission periods, the emission periods of different fragrances may vary. For example, if a 24 hour emission cycle is utilized, a first fragrance may be emitted for 6 hours, a second for 8 hours, and a third for 7 hours, followed by a 3 hour period of no emission.

Still further, one or more of the fragrances of the second mode 300 may be emitted for a random period of time such that the emission periods for the fragrances and the period of no emission equal a predetermined emission cycle. In particular, a random number generator may be utilized to determine one or more emission periods and/or the period of no emission, wherein the random number determines either an emission period in a selected range, such as those disclosed above, or a percentage within a selected range, such as the ranges discussed above. The random percentage would then be multiplied by the emission cycle to determine the appropriate emission period or period of no emission. Random percentages and/or random emission periods may be determined using methods and equations similar to the methods and equations for randomizing emission periods described in one or more of Gasper U.S. application Ser. No. 12/565,530, filed on Sep. 23, 2009, and entitled “Method of Dispensing a Volatile Material” or Gasper U.S. application Ser. No. 12/565,550, filed on Sep. 23, 2009, and entitled “Method of Dispensing a Volatile Material,” the disclosures of which are incorporated herein in their entireties.

Employing an emission cycle of 24 hours, two or more fragrances may each be emitted in the second mode 300 for emission periods of between about 3 hours and about 12 hours, more preferably between about 5 hours and about 10 hours, and most preferably about 7 hours. The fragrances are emitted with a period of low or no fragrance emission of between about 0 hours and about 10 hours, more preferably between about 1 hour and about 5 hours, and most preferably about 3 hours or about 4 hours.

FIG. 10B illustrates a diagram representing a sample execution of an implementation of the second mode 300 of dispensing described hereinabove with regard to FIG. 10A. Block 350 represents a DC power supply, for example, a rechargeable battery pack that provides uninterrupted power to block 352, which represents a mechanism that maintains the real time. In the case of an AC power failure, the current time is updated via the blocks 352 and 354 after the AC power has been restored at block 356.

After AC power has been supplied to the device, a determination is made utilizing the current time at block 358 whether the current time is, for example, between 6:00 am and 12:00 pm. If the current time is within this range, the first fragrance 302 is emitted for a first period of time at block 360. A first loop timer continues to compare the current time against the time range of the block 358 to determine whether the first fragrance 302 should still be emitted. In such scenario, the dispensing at the block 360 would only occur for the first period of time, for example, 15 minutes, or any other desired period of time less than the time range represented by the block 358. Optionally, the first loop timer could be removed and the dispensing at the block 360 could be set to the same period as represented by the block 358, for example, 6 hours.

After the current time reaches 12:00 pm, the block 362 utilizes the current time to determine whether the current time is, for example, between 12:00 pm and 6:00 pm. If so, a second fragrance 304 is emitted for a second period of time at block 364. A second loop timer continues to compare the current time against the time range of the block 362 to determine whether to continue dispensing the second fragrance 304. After the current time reaches 6:00 pm, the block 366 utilizes the current time to determine whether the current time is, for example, between 6:00 pm and 12:00 am. If so, the third fragrance 306 is emitted for a third period of time at block 368. A third loop timer continues to compare the current time against the time range of the block 366 to determine whether to continue dispensing the third fragrance 306. As with the first loop timer the second and third periods may be any period of time less than the time ranges of the blocks 362 and 366, respectively. Also optionally, the second and/or third loop timers may be removed such that the second and third fragrances 304, 306 would be emitted for the time range of the blocks 362, 366, respectively.

After the current time reaches 12:00 am, block 370 utilizes the current time to determine whether the current time is, for example, between 12:00 am and 6:00 am. If so, block 372 executes and no fragrance is emitted. A fourth loop timer continues to compare the current time against the time range of the block 370 to determine whether no fragrance should still be emitted. After the current time reaches, for example, 6:00 am, operation returns to the block 358.

Illustrative examples of fragrances that may be used in the second mode 300 of dispensing include, but are not limited to, a “Lemon Chamomile” fragrance for the morning period, a “Clean Linen” fragrance for the afternoon period, and a “Lavender Vanilla” fragrance for the evening period. Each of the fragrances described by name herein is sold under the Glade® trademark by S.C. Johnson & Son, Inc., of Racine, Wis.

An apparatus that executes the second mode 300 of dispensing includes an internal clock or timer to allow the apparatus to execute changes in the dispensed fragrance at the appropriate times during the day. It is contemplated that such execution could be realized automatically by inclusion of an internal clock that may be a variety of “atomic clock” that links by radio signal to synchronize with an official clock, for example, the official U.S. atomic clock located in Boulder, Colo. Alternatively, an automatic interface utilizing light, temperature, and/or activity sensors and/or appropriate software or firmware may be utilized to customize the apparatus to accommodate a user's daily routine as a “smart” device. (For example, the apparatus would sense the time of day the user generally leaves home and the time of day the user generally returns home and would adjust the periods of emission to match the user's routine.)

Still further, a visual interface could be included that allows the user to enter commands manually. The visual interface could include, but is not limited to a clock display having a manual keypad for data entry or a simple set button that operates with, for example, light emitting diode (LED) or liquid crystal display (LCD) indicators that blink while being set. The user may enter a selection for a living space, for example, living room, bedroom, bathroom, utility room, dining room, kitchen, etc., where the selected living space is used by the smart device to further adjust the periods of emission as appropriate. Still alternatively, different emission programs may be implemented depending on a living space selection. For example, if “bedroom” is selected, the device may execute a program such as that described with regard to FIGS. 10A and 10B. Alternatively, if “bathroom” is selected, the device may only emit fragrance when a user's presence or a light is sensed.

In regard to the second mode 300 of dispensing, three empirical studies have demonstrated that the second mode 300 of dispensing fragrances including a night dwell period is more effective in inhibiting adaptation and/or habituation than a test sequence having 45 minute dispensing periods of similar fragrances (hereinafter “the 45 minute sequence”). Identical study apparatuses were constructed for the 45 minute sequence and the second mode 300 to dispense two or three fragrances, respectively, according to the appropriate algorithm. The study apparatuses utilized for the 45 minute sequence and the second mode 300 included two or three power outlets, respectively, with programmable timers. Single fragrance dispensers each with a container of scented oil were plugged into the appropriate number of power outlets and the dispensers were operated according to a controller that ran either the 45 minute sequence or the second mode 300 of operation. The single fragrance dispenser used in the first empirical study is detailed in Zobele U.S. Pat. No. 6,996,335.

In a first study, the 45 minute sequence and a first study implementation of the second mode 300 of dispensing seen in FIG. 10A were compared using the study apparatuses described hereinabove for effectiveness of adaptation and/or habituation inhibition by a first group of twenty-five women over about a two week period. The first study implementation of the second mode 300 of dispensing included a 6 hour morning period during which a “Ferns and Blossoms” fragrance was dispensed followed by a 6 hour afternoon period during which the “Clean Linen” fragrance was dispensed. Subsequently, a “White Tea and Lily” fragrance was dispensed for a 6 hour evening period followed by a 6 hour night dwell period during which no fragrance was dispensed. The 45 minute sequence was a repeating sequence in which the “Ferns and Blossoms” fragrance was dispensed for 45 minutes followed by 45 minutes of the “Clean Linen” fragrance followed by a 45 minute dwell period. Responses collected from the first group after a first study period of about two weeks indicate that the above-noted implementation of the second mode 300 of dispensing including 6 hour dispensing periods was more effective in inhibiting adaptation and/or habituation than the 45 minute sequence for every category of fragrance perception studied. The categories include: how well the fragrance was smelled when the user first walked into the room, how often the fragrance was noticed when the user was across the room from the dispenser, how often the fragrance was noticed after the user was in the room for an extended period of time, how well the fragrance freshened the room throughout the day, how often the user caught a whiff of the fragrance when they were not thinking about it, how well the user could smell the fragrance when the user thought about it and tried to smell it, and how strong the fragrance was overall.

In a second study, the 45 minute sequence and a second study implementation of the second mode 300 of dispensing were compared for effectiveness of adaptation and/or habituation inhibition by a second group of twenty-three trained personnel over about a 2 week period. The study personnel included persons trained in scent perception. The second study utilized the same study apparatuses and single fragrance dispensers as were used in the first study.

The second study implementation of the second mode 300 of dispensing included a 6.5 hour morning period between 5:00 am and 11:30 am during which a “Sunny Days” fragrance was dispensed followed by a 6.5 hour afternoon period between 11:30 am and 6 pm during which the “Clean Linen” fragrance was dispensed. Subsequently, a “Lavender Meadow” fragrance was dispensed for a 6.5 hour evening period between 6 pm and 12:30 am followed by a 4.5 hour night dwell period between 12:30 am and 5 am during which no fragrance was dispensed. The 45 minute sequence was a repeating sequence in which the “Clean Linen” fragrance was dispensed for 45 minutes followed by 45 minutes of the “Sunny Days” fragrance followed by a 45 minute dwell period.

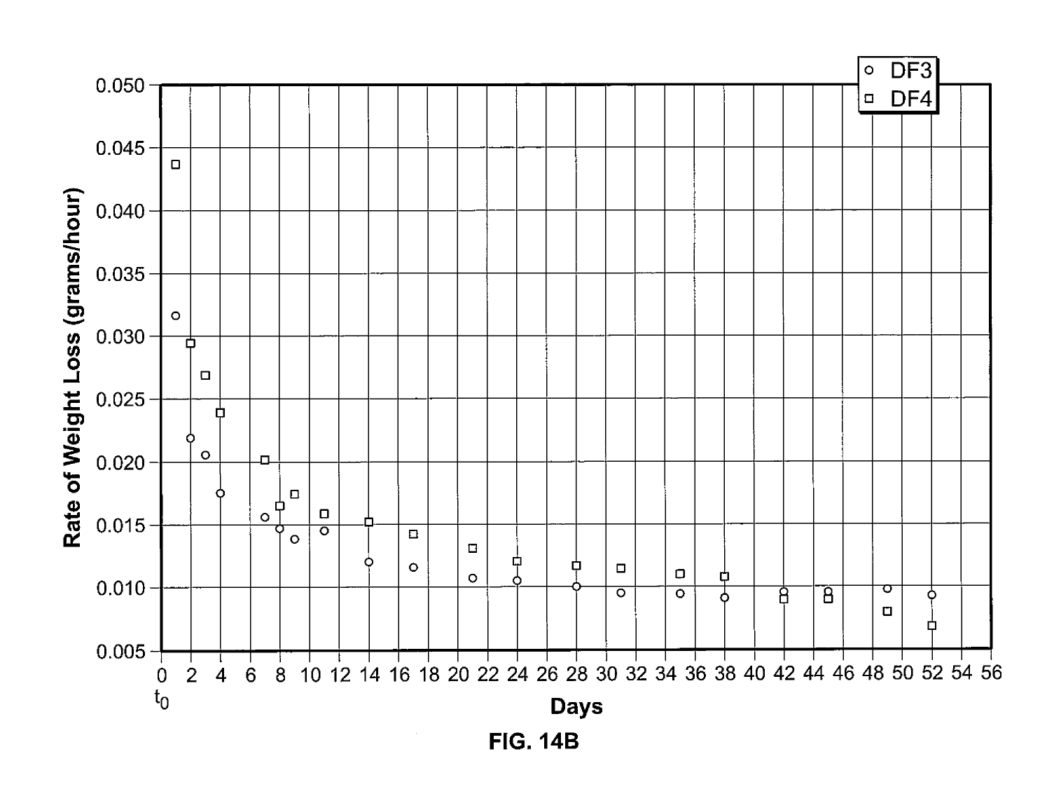

Each of the twenty-three trained personnel was asked to evaluate the perceived intensity of fragrance four times a day, between 7 am and 9 am, between 3 pm and 5 pm, between 7:30 pm and 9 pm, and between 10 pm and midnight. The evaluations were made on a scale from 0 to 15, where 0 corresponds to no fragrance detected and 15 corresponds to a very strong fragrance detected. Responses from the twenty-three trained personnel over a second study period spanning sixty-two evaluations (about 15.5 days) were averaged and the averaged response data for each evaluation are shown in the graph of FIG. 11. The response data indicates that the second study implementation of the second mode 300 of dispensing demonstrated a significantly higher strength of perception of fragrance throughout the second study period than the 45 minute sequence.

In a third study implementation of the second mode 300 of dispensing, 25 women (“the respondents”) were given test units for a six-week in-home use test. Every two weeks, the respondents were given a different test product and the respondents were asked to keep a journal of their experiences with each test product and, at the end of each two weeks, completed a questionnaire for the given product. The test units included a test box with three outlets and programmable timers corresponding to each of the outlets. In the first two-week test, the first test product included a single plug-in scented oil dispenser plugged into one of the three outlets. The single dispenser used in the first, second, and third tests is detailed in Zobele U.S. Pat. No. 6,996,335. The single dispenser included a “Clean Linen” fragrance, wherein the single dispenser was continuously activated for the entirety of the two-week test. During the second two-week test, the second test product included three dispensers plugged into the three outlets. A first of the dispensers included a “Ferns & Blossoms” fragrance, wherein the first dispenser was actuated for 6 hours during the morning hours, the second of the dispensers included the “Clean Linen” fragrance, wherein the second dispenser was actuated for 6 hours during the afternoon hours, and the third of the dispensers included a “White Tea & Lily” fragrance, wherein the third dispenser was actuated for 6 hours during the evening hours. Actuation of the three dispensers was then followed by a 6 hour off period in which no dispenser was actuated. Each of the programmable timers was pre-programmed to operate a respective dispenser at the appropriate hours with the pattern repeating every 24 hours for two weeks. During the third two-week test, the third test product included three dispensers plugged into the three outlets. A first of the dispensers included the “Ferns & Blossoms” fragrance, a second of the dispensers included a “Clean Linen” fragrance, and a third of the dispensers included no fragrance (which acted as a placebo). The programmable timers for the two utilized outlets were pre-programmed to actuate the dispensers in an alternating 45-minute sequence. Specifically, the first dispenser was operated for 45 minutes and, at the same time the first dispenser was deactivated, the second dispenser was actuated for 45 minutes, and thereafter, at the same time the second dispenser was deactivated, the first dispenser was again actuated for 45 minutes and the pattern repeated continuously for two weeks.

Some of the answers gathered from the respondents were averaged and are recorded in Table 1 below, wherein the respondents replied with ratings ranging between a rating of 1 corresponding to an extreme dislike or no inability to smell the fragrance to a rating of 9 corresponding to an extreme liking or ability to smell the fragrance:

TABLE 1

| | | Second Test Product (three | Third Test Product (two |
|---|---|---|---|
| | First Test Product | dispensers for 6 hours each | dispensers alternated every |
| | (single dispenser) | followed by 6 hours off) | 45 minutes) |
| How well could you smell | 6.4 | 7.2 | 6.1 |
| the fragrance when you | | | |
| walked into the room? | | | |
| How often did you notice | 5.4 | 6.2 | 4.8 |
| the fragrance when you | | | |
| were across the room from | | | |
| the unit? | | | |
| How often did you notice | 5.0 | 6.7 | 5.2 |
| the fragrance after you | | | |
| were in the room for an | | | |
| extended period? | | | |
| How well did the | 6.1 | 7.1 | 5.9 |
| fragrance freshen the room | | | |
| throughout the day? | | | |
| How often did you catch a | 5.8 | 6.5 | 5.2 |
| whiff of the fragrance | | | |
| when you weren't thinking | | | |
| about it? | | | |
| How well could you smell | 6.6 | 7.3 | 6.6 |
| the fragrance when you | | | |
| thought about it and tried | | | |
| to smell it? | | | |
| How strong was the | 5.6 | 6.4 | 5.4 |
| overall fragrance? | | | |

From Table 1, it can be seen that there was an overall greater noticeability of fragrance when the second test product was utilized, as opposed to the first and third test products.

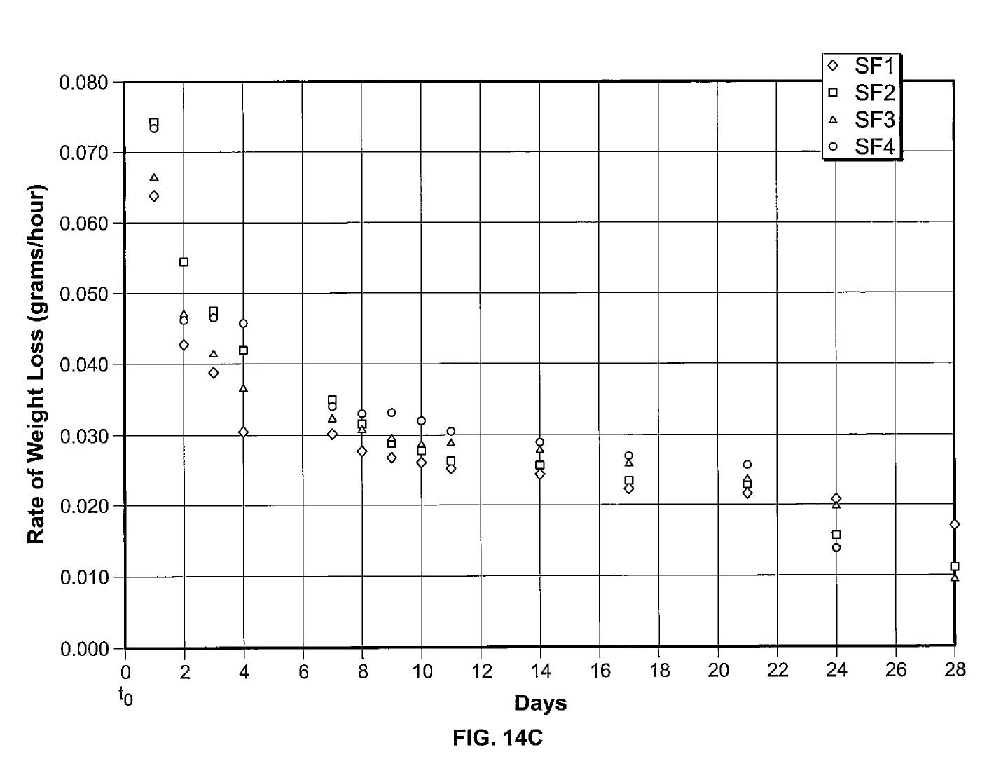

A third mode 400 of dispensing, illustrated in FIGS. 12A and 12B, includes two or more fragrances sequentially dispensed with one of the two or more fragrances being perceived as highly efficacious for odor elimination and the other of the two or more fragrances being perceived as having a more subtle fresh scent. For example, first and second subtle fresh scents 402, 404 are dispensed sequentially during daylight hours, or between about 6 a.m. and 10 p.m., to give a user the feeling of fresh air. Although the first and second scents 402, 404 are depicted as being sequentially dispensed, such scents may be continuously alternated throughout the daylight hours, for example for periods of 45 minutes or more. An odor eliminating fragrance 406 is dispensed during the night, for example, between about 10 p.m. and 6 a.m., to give the user the feeling that the air is being cleaned of odors while they sleep. As illustrated in FIG. 12B, the first and second subtle fresh scents 402, 404 and the odor eliminating scent 406 may be dispensed with approximately equal concentrations or intensities, or alternatively as seen in FIG. 12A, the first and second subtle fresh scents 402, 404 may be dispensed at a relatively weak concentration or intensity and the odor eliminating fragrance 406 may be dispensed with a relatively strong concentration or intensity. Although two subtle fresh scents 402, 404 are depicted in the example implementations of the third mode 400, only one fresh scent is necessary. Further, any number of fresh scents may be utilized. Illustrative examples of fragrances that may be perceived as highly efficacious for odor elimination include, but are not limited to mint, bleach, and citrus.

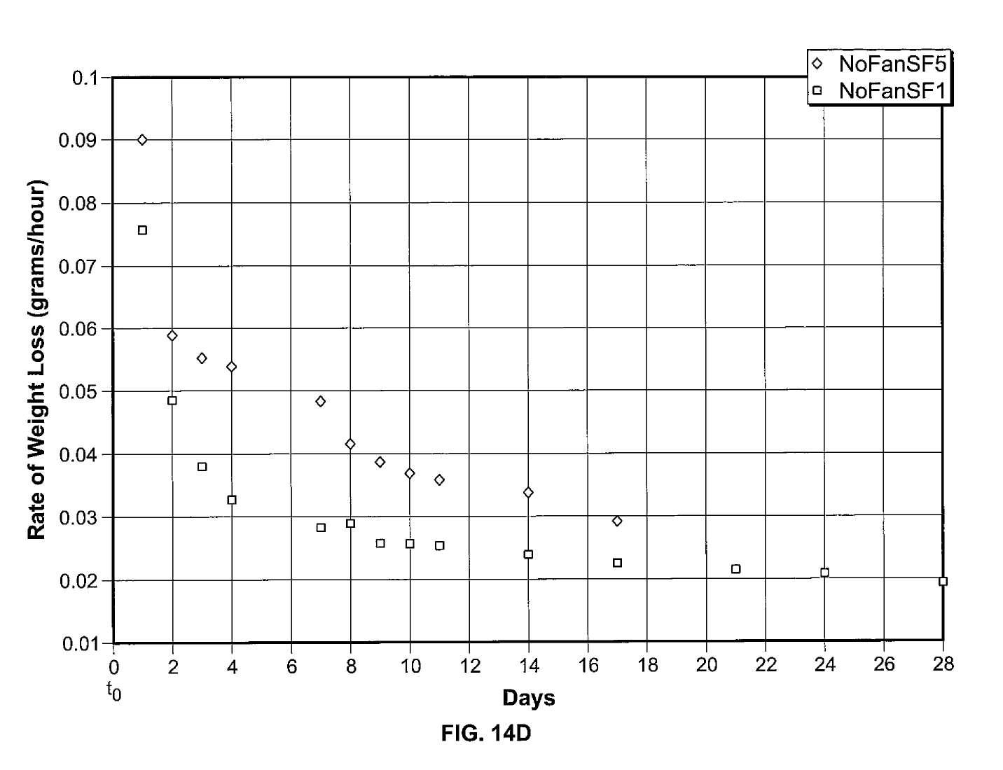

In a fourth mode 500 of dispensing, two or more fragrances are alternatingly emitted with a gap therebetween, wherein during the gap, a fragrance intended to cleanse the olfactory receptors of a user is dispensed to allow the user to take full advantage of each of the two or more fragrances. For example, referring to FIGS. 13A and 13B, a palate cleansing fragrance 502 is dispensed in shorter time periods between longer time periods during which first and second intended fragrances 504, 506 are dispensed. For example, the first and second intended fragrances may be alternatingly dispersed for about an hour each, separated by the palate cleansing fragrance 502, which is dispersed for 15 minutes. Relative intensities or concentrations of the first and second intended fragrances 504, 506 and the palate cleansing fragrance 502 may be equal, as illustrated in FIG. 13B or unequal, as illustrated in FIG. 13A. Illustrative examples of the palate cleansing fragrances 502 include, but are not limited to, coffee bean, lemon, and clean cotton.

It is a known phenomenon that the rate of dispensing of a volatile material dispensed from a volatile material dispenser operating in a steady state condition may decay over time. As disclosed by Kvietok et al. U.S. Pat. No. 7,481,380, the rate of dispensing may decay for any of a number of reasons including, but not limited to a clogged or fouled emanator surface or wick, a loss of volatility of the volatile material over time due to more volatile components of the volatile material volatilizing before less volatile components thereof, and/or other similar reasons. In fact, the rate of dispensing typically has a decay profile that may be a function of the particular dispenser, the device(s) utilized to dispense the volatile material (e.g., fan, heater, piezoelectric device, and/or other devices known in the art), the type of the volatile material (e.g., a fragrance, an aromatherapy scent, a positive fragrancing active material, or an air-freshener), and/or the form of the volatile material (e.g., scented oil, solid, or gel).

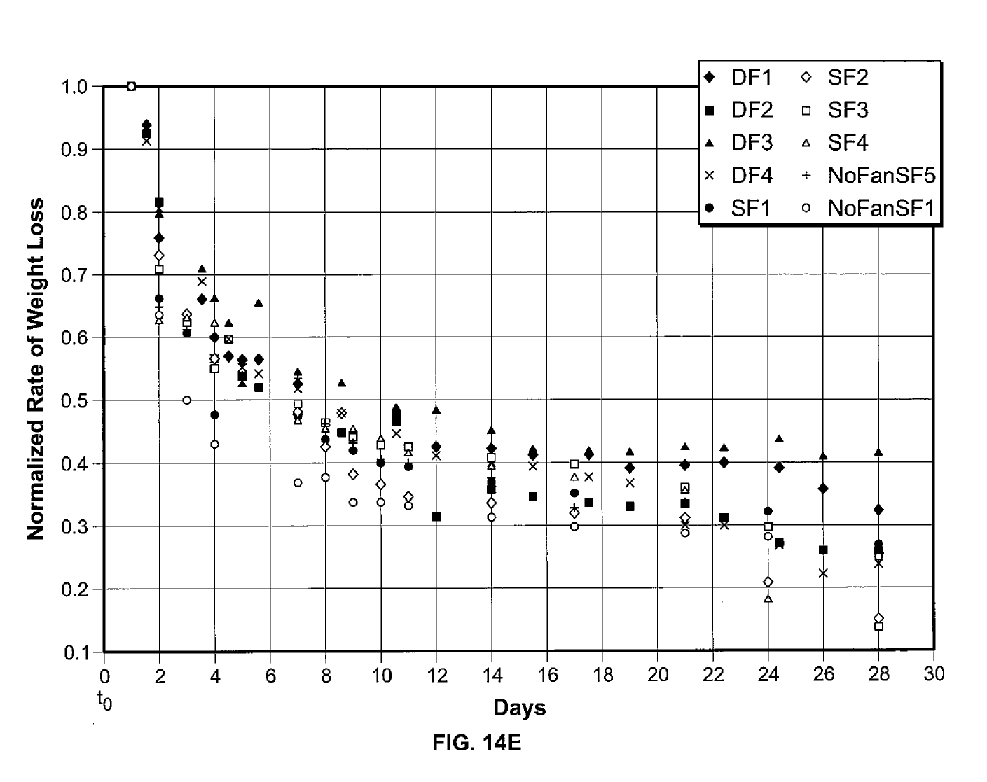

A common way to quantify the rate of dispensing of volatile material from a volatile material dispenser is to measure the rate of weight loss over time of the volatile material dispenser operating in a steady state condition. A first test was conducted to gather a first set of measurements to produce the data seen in FIG. 14A. The first test was performed using dual fragrance dispensers having a heater associated with each fragrance and a fan. Each of the dual fragrance dispensers included a left compartment and a right compartment, wherein each compartment can accommodate a container of scented oil fragrance. Each container included a wick disposed in contact with a fragrance disposed in the container, wherein the wick extends through a top portion of the container. When disposed within the left and right compartments, the wicks extending from the containers were disposed adjacent the heaters disposed in the respective compartments. The dual fragrance dispenser used in the first test is detailed in FIGS. 25-28 and paragraphs [0064]-[0073] in Porchia et al. U.S. patent application Ser. No. 12/288,606. In the first test, six samples were tested for each of two fragrances F₁, F₂, where F₁ was the “Clean Linen” fragrance and F₂ was the “Ferns and Blossoms” fragrance. Three samples of each fragrance F₁, F₂ were tested in the left compartment and three samples of each fragrance F₁, F₂ were tested in the right compartment. Accordingly, one sample of each fragrance F₁, F₂ was weighed and loaded into each of six dispensers. The samples for each dispenser were balanced such that the total fragrance weight disposed in the container placed into the right compartment was the same as the total fragrance weight disposed in the container placed into the left compartment. Each dispenser was subsequently plugged into a power outlet and placed in a maximum power setting. Each dispenser operated by generally alternatingly heating the wicks extending from the containers and disposed in the left and right compartments for 45 minutes each. Each sample was weighed daily for the first ten working days of the test and subsequently twice a week until the six samples for each fragrance F₁, F₂ reached an endpoint designated as a point in time when all but 2 grams of the fragrance was exhausted based on an average weight of the six samples.

The weight measurements taken during the first test were averaged for each fragrance F₁ and F₂ across the six samples. Differences in the measured weights were divided by the intervening time intervals between the measurements to yield average rates of weight loss in grams/hour that are plotted in the graph of FIG. 14A. For example, the weight of fragrance F₁ weighed at day 1 was subtracted from the weight of fragrance F₁ weighed at day 0 (time t₀) to yield a difference in weight between days 0 and 1. The difference in weight was divided by 24 hours to yield an average rate of weight loss in grams/hour, which is then depicted as the data point at day 1. The data plotted in FIG. 14A illustrate decay profiles for the rates of weight loss for the fragrances F₁ and F₂ dispensed by dual fragrance dispensers with heaters and a fan. Because dual dispensers were used in the first test (and the second test described hereinbelow), the rates of weight loss are representative of heating the wicks for about half of the overall time the dispenser is active (due to the alternation of two fragrances). The data points in the graph of FIG. 14A are labeled as DF₁ and DF₂ to indicate that the fragrances F₁ and F₂, respectively, were dispensed by a dual fragrance dispenser with heaters and a fan. The decay profiles illustrated in FIG. 14A will be described in greater detail hereinbelow.

A second test was performed using dual fragrance dispensers that were identical in structure and function to the dual fragrance dispensers described hereinabove with regard to FIG. 14A. Six samples were again tested for each of two fragrances F₃ and F₄ using an identical methodology to that described hereinabove for the fragrances F₁ and F₂. The fragrance F₃ was a “Hawaiian Breeze” fragrance and the fragrance F₄ was a “Vanilla & Cream” fragrance. Computed rates of weight loss (grams/hour) for the fragrances F₃ and F₄ are plotted in the graph of FIG. 14B. The data plotted in FIG. 14B illustrate decay profiles for the rates of weight loss for the fragrances F₃ and F₄ dispensed by dual fragrance dispensers with heaters and a fan. The data points in the graph of FIG. 14B are therefore labeled as DF₃ and DF₄ to indicate that the fragrances F₃ and F₄, respectively, were dispensed by a dual fragrance dispenser with heaters and a fan. The first data point is displayed at day 1 for each fragrance F₃, F₄ and is a measure of the rate of weight loss between an initial time t₀ and day 1. The decay profiles illustrated in FIG. 14B will be described in greater detail hereinbelow.

A third test was conducted using single fragrance dispensers having a heater and a fan. Each of the single fragrance dispensers accommodate a single container of scented oil fragrance having a wick in contact with a fragrance disposed in the container, wherein the wick extends through a top portion of the container. When disposed within the dispenser, the wick was disposed adjacent a heater disposed in the dispenser. The single fragrance dispenser used in the third set of measurements is detailed in Pedrotti et al. U.S. Pat. No. 6,862,403. One sample of each of the fragrances F₁, F₂, F₃, and F₄ was weighed and loaded into each of four single fragrance dispensers. Each dispenser was subsequently plugged into a power outlet and placed in a maximum power setting. While the dispensers were operating, the heater and the fan ran continuously. Each sample was weighed daily for the first ten working days of the test and subsequently twice a week until the sample reached an endpoint designated as a point in time when all but 2 grams of the fragrance was exhausted based on an average weight of the four samples.

Differences in the measured weights were divided by the intervening time intervals between the measurements to yield rates of weight loss (grams/hour) that are plotted in the graph of FIG. 14C. The data plotted in FIG. 14C illustrate decay profiles for the rates of weight loss for the fragrances F₁, F₂, F₃, and F₄ dispensed by a single fragrance dispenser with a heater and a fan. The data points for each of the fragrances F₁, F₂, F₃, F₄ are therefore designated on the graph of FIG. 14C as SF₁, SF₂, SF₃, SF₄, respectively, to distinguish the data acquired using a single fragrance dispenser including a heater and a fan from the data acquired using the dual fragrance dispensers described hereinabove with regard to FIGS. 14A and 14B. As noted hereinabove, the first data point is displayed at day 1 for each fragrance F₁, F₂, F₃, F₄ and is a measure of the rate of weight loss between an initial time tₒ and day 1. The decay profiles illustrated in FIG. 14C will be described in greater detail hereinbelow.

A fourth test performed using single fragrance dispensers with only a heater (no fan). Containers of a scented oil fragrance F₅ (a “Vanilla Breeze” fragrance) and the above-noted fragrance F₁ were tested. Each container included a wick in contact with a fragrance disposed in the container, wherein the wick extends through a top portion of the container. When disposed within the dispenser, the wick is in proximity to the heater. The single fragrance dispenser used in the fourth set of measurements is detailed in Zobele U.S. Pat. No. 6,996,335. Six samples were tested for each of the fragrances F₅, F₁. One sample of each of the fragrances F₅, F₁ was weighed and loaded into each single fragrance dispenser. Each dispenser was subsequently plugged into a power outlet and placed in a maximum power setting. During operation of each dispenser, the heater was energized continuously, thus heating the wick of the container held therein. Each sample was weighed daily for the first ten working days of the test and subsequently twice a week until the samples reached an endpoint designated as a point in time when all but 2 grams of the fragrance was exhausted based on an average weight of the six samples.

Differences in the measured weights were divided by the intervening time intervals between the measurements to yield rates of weight loss (grams/hour) that are plotted in the graph of FIG. 14D. The data plotted in FIG. 14D illustrate decay profiles for the rates of weight loss for the fragrances F₅ and F₁ dispensed by the single fragrance dispensers with only a heater. The data points for each of the fragrances F₅, F₁ are therefore designated on the graph of FIG. 14D as NoFanSF₅, NoFanSF₁, respectively, to distinguish the data acquired using a single fragrance dispenser with only a heater from the data acquired using the single fragrance dispensers that include a heater and a fan, as described hereinabove with regard to FIG. 14C. As noted hereinabove, the first data point is displayed at day 1 for each fragrance F₅, F₁ and is a measure of the rate of weight loss between an initial time t₀ and day 1. The decay profiles illustrated in FIG. 14D will be described in greater detail hereinbelow.

Referring to the graphs of data in FIGS. 14A-14D, each of the fragrances has a somewhat different profile for rate of weight loss over time. For example, referring to FIG. 14A, the rates of weight loss for DF₁ are substantially lower than the rates of weight loss for DF₂ over at least the first ten days. Also, the rates of weight loss represented in the graphs of FIGS. 14A and 14B are about half of the rates of weight loss represented in the graphs of FIGS. 14C and 14D because the former are the results of a dual fragrance dispenser that alternates between fragrances and the latter are the results of a single fragrance dispenser that is on continuously. However, the trends indicated in each of the graphs are similar, which indicates that the behavior of the rate of weight loss over time follows a qualitative pattern. For example, the rate of weight loss of each of the fragrances discussed hereinabove is greatest at the beginning of the dispensing period as reflected by the rate data point at day 1. The rate of weight loss decreases rapidly from the first day until about the seventh day and subsequently decreases less rapidly.

The decay in the rate of weight loss of a fragrance dispenser operating in steady state conditions, as seen in FIGS. 14A-14D, corresponds with a decay in the amount of fragrance emitted, and a decay in a fragrance intensity perceived by a user also corresponds with the decay in the amount of fragrance emitted. Therefore, the fragrance intensity perceived by a user corresponds with a decay in the rate of weight loss of a fragrance dispenser. Reduced perception by the user would likely have a negative effect on efforts to minimize adaptation and/or habituation of the user to the fragrance, as described hereinabove. A mathematical quantification of the observed qualitative trend indicated by the data in the graphs of FIGS. 14A-14D may be useful in a method of fighting adaptation and/or habituation.

The data as presented in the graphs of FIGS. 14A-14D for each of the tests have substantially different values and span different time periods, which makes a comparison between the qualitative trends rather difficult. As a first step in a comparison of the observed qualitative trends, it would be helpful to be able to compare the data in the graphs of FIGS. 14A-14D on an apples to apples basis. A common way to achieve such a comparison is to manipulate all of the data so that each data set shares a corresponding point of identical magnitude and spans an equalized time period.

For example, it was noted hereinabove that the data sets for the first and second tests, the data for which are depicted in FIGS. 14A and 14B, represent rates of weight loss measured over time intervals that are about twice the time intervals that heating was actually applied because the dual fragrance dispenser alternates heating between the fragrances. If the rates of weight loss for the first and second sets of data were recomputed using actual time of heating, a better comparison could be made with the rates of weight loss for the third and fourth sets of data. Such a “time normalization” procedure establishes a common basis for a determination of the rate of weight loss for all of the data sets. In practice, the measured weights of the fragrances in the first and second tests are differenced and then divided by half of the actual time interval between measurements to yield time normalized rates of weight loss for the first and second tests.

Further, each of the data sets could be divided by the rate of weight loss as computed at about day 1. Such an “endpoint normalization” procedure establishes a corresponding starting data point at day 1 of magnitude 1 for each data set. Having a common starting data point and a common time normalized interval thus allows the decay profiles of all the data sets to be meaningfully compared. The data sets illustrated in the graphs of FIGS. 14A-14D were normalized by applying the time normalization procedure on the data in FIGS. 14A and 14B and by applying the endpoint normalization procedure on the data in all of the FIGS. 14A-14D. The results of the fully normalized data sets are illustrated in the graph of FIG. 14E. Because the normalization procedures employed herein are rather crude and the size of the data sets are statistically small, the slight spread in the data is expected. Nevertheless, FIG. 14E illustrates that the decay profiles of FIGS. 14A-14D when fully normalized are, in fact, similar.

The normalized data illustrated in FIG. 14E can be further manipulated to facilitate analysis by taking an average of the data across all of the data sets. FIG. 14F illustrates a graph of an average normalized rate of weight loss profile, wherein the normalized profiles illustrated in FIG. 14E have been averaged. The averaged normalized data in FIG. 14F can be integrated over time to yield a total normalized weight of dispensed fragrance and can be likewise integrated over a segment of time to yield the normalized weight of fragrance dispensed during the segment. Using this method of integration, one can determine how long it would take to dispense a fraction of the total normalized weight of dispensed fragrance.

The average normalized rate of weight loss profile illustrated in FIG. 14F may be used to develop a method for inhibiting adaptation and/or habituation by a user. One may define a normalized characteristic time decay period T_{c} that, for example, may represent the amount of time required for the averaged normalized data presented in FIG. 14F to decrease from an initial normalized rate of weight loss (as computed at the end of day 1) to a normalized rate of weight loss that is a predetermined fraction of the initial normalized rate of weight loss. Similarly, one may define a characteristic time decay period T_{c} that, for example, may represent the amount of time required for a fragrance dispenser (or any volatile material dispenser) operating in steady state conditions to reach a rate of weight loss that is a predetermined fraction of the initial rate of weight loss (as computed at the end of day 1). A dispenser that dispenses fragrance for a time that is less than or equal to T_{c} may have an increased effectiveness as part of an effort to minimize adaptation and/or habituation over a dispenser that dispenses the volatile material for a time greater than T_{c}.

The predetermined fraction that is chosen may depend upon practical considerations such as container size, the time a container lasts before being exhausted, and the number of containers that can be accommodated by a dispenser. From the standpoint of inhibiting adaptation and/or habituation, the container or compartment size that is most useful is the size having the highest possible rate of weight loss. Therefore a large number of very small containers or compartments would be desirable, where each very small container or compartment lasts a short time but has a high rate of weight loss. However, from the standpoint of user convenience and manufacturing cost, fewer containers or compartments each lasting a longer time would be desirable. A compromise may be made between the highest possible rate of weight loss and a reasonable number of containers or compartments each lasting a reasonable length of time by examining the data in FIG. 14F.

Referring to FIG. 14F, the average normalized rate of weight loss profile has a rate of weight loss at about day 7 that is about one half of the rate of weight loss as compared to an initial rate of weight loss. Therefore, after about day 7, each of the volatile material dispensers is dispensing volatile material into the environment at a normalized rate that is about one half of an initial normalized rate. Still referring to FIG. 14F, subsequent to about day 7, each of the normalized profiles continues to decay but at a decreased rate of decay such that, for example, at about day 14 the normalized rate of weight loss is about 0.4 of the of the initial normalized rate and at about day 21 the normalized rate of weight loss is about 0.35 of the initial normalized rate. Therefore, after about day 7, the amount of fragrance dispensed has decreased dramatically, and thus, a user's perception of such fragrance has also likely decreased.

Assuming that a typical container of scented oil may last about twenty-eight days of continuous use, integration of the data shown in FIG. 14F indicates that a container having about 50% of the volume of the typical container would last about 10 days due to the front loaded effect of the decay profile. Therefore, a dispenser that could accommodate three containers or a cartridge having three compartments each having about 50% of the volume of the typical container would operate about 30 days before being exhausted and would dispense fragrance at a normalized rate of weight loss of greater than between about 0.4 and about 0.45 during the entire 30 day period. Thus, T_{c} defined for a predetermined fraction equal to between about 0.4 and about 0.45 has a period of about 10 days.

Similarly, integration of the data shown in FIG. 14F indicates that a container that would last about 7 days would have about 40% of the volume of the typical container. Four such containers or a cartridge having four such compartments would be required to last 28 days; however, the fragrance dispenser would dispense fragrance at a normalized rate of weight loss of greater than about 0.5 during the entire 28 day period. Thus, T_{c} defined for a predetermined fraction equal to about 0.5 has a period of about 7 days.

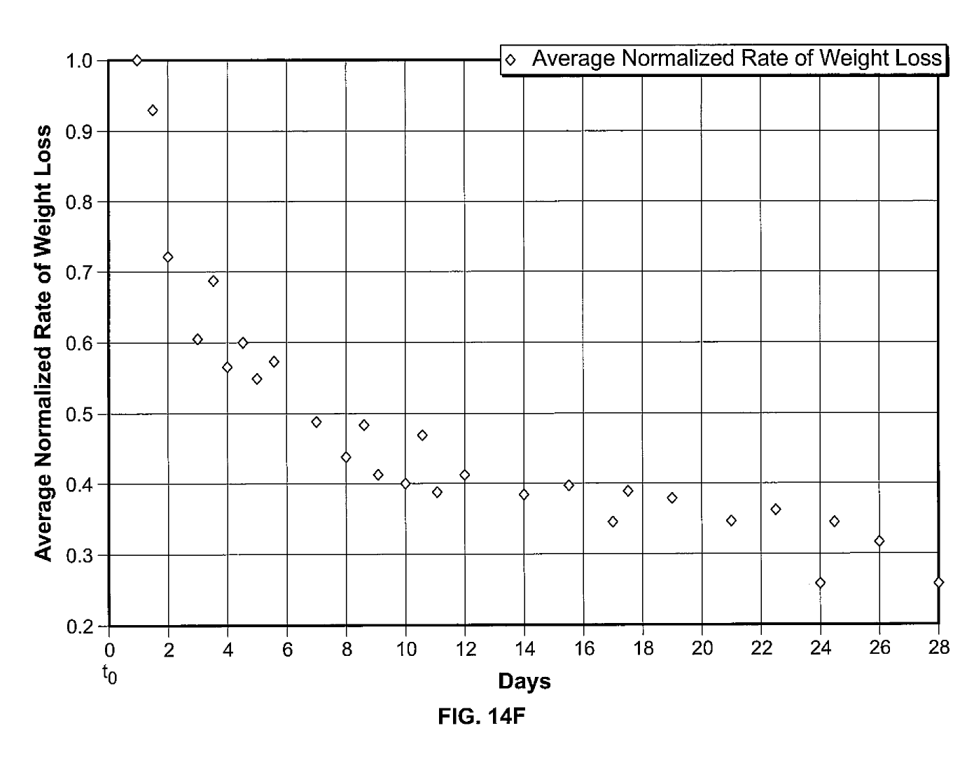

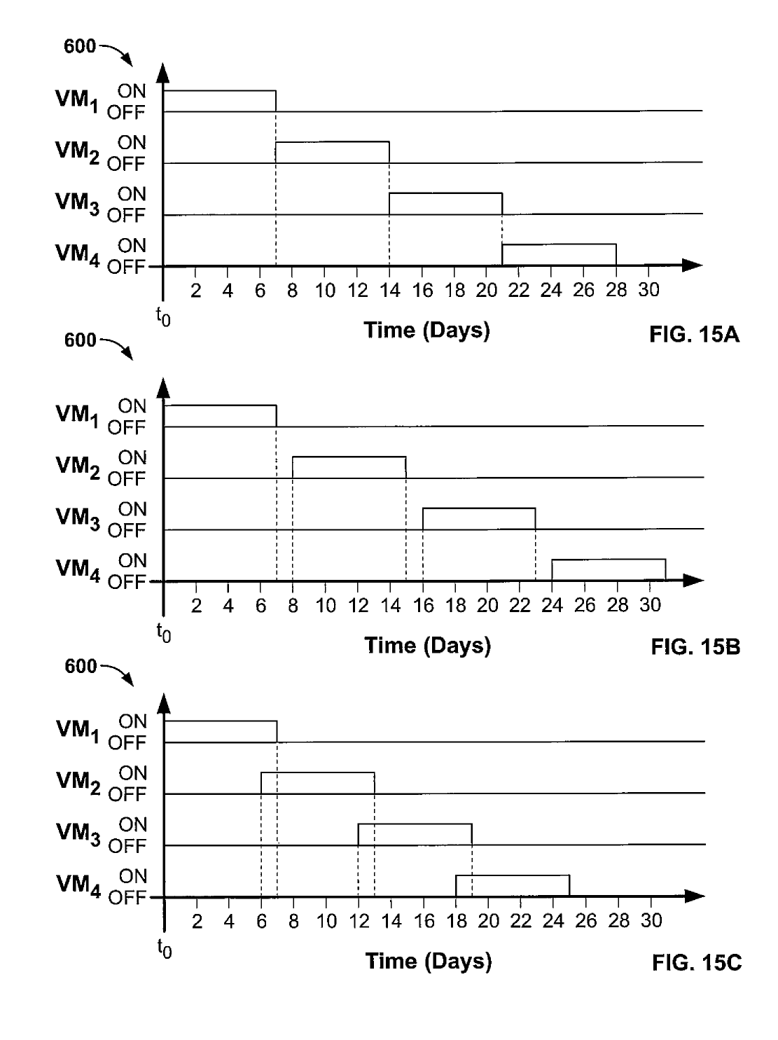

In view of the foregoing, a fifth mode 600 of dispensing, as illustrated in FIG. 15A, has been developed and includes repeated shortened periods of dispensing of the same fragrance from different fragrance containers or a cartridge having different fragrance compartments, wherein the shortened periods of dispensing are tailored to be shorter than or about equal in time to the characteristic time decay period T_{c}. Each such shortened period of dispensing is operative on a separate container or cartridge of the volatile material that lasts for about the characteristic time decay period T_{c} before being exhausted. FIG. 16 illustrates a normalized rate of weight loss decay profile for such containers utilized by the fifth mode 600 of dispensing. For illustrative use in FIG. 16, the characteristic time decay period T_{c} is defined as the time it takes for the normalized rate of weight loss to reach about 0.5 of an initial rate, which is about 7 days for the test results presented herein. The fifth mode 600 of dispensing, as illustrated by FIGS. 15A and 16, eliminates the typical decay profile for the normalized rate of weight loss of the fragrance beyond the characteristic time T_{c}, during which time the average rate of weight loss is significantly less than before the characteristic time Tₑ. Therefore, the fifth mode 600 of dispensing may be useful in decreasing and/or diminishing adaptation and/or habituation and may waste less volatile material because the amount of volatile material dispensed after the characteristic time decay period T_{c} is eliminated.

The fifth mode 600 of dispensing may include periods of dispensing that may or may not be separated by a dwell period or that may overlap. During the periods of dispensing, the individual volatile materials are continuously emitted. By continuous, it is meant that a dispensed volatile material is emitted for the entire period of dispensing, although the dispensing may be intermittent. For example, if a heater is utilized, the heater may be pulse-width modulated during such continuous emission period or if an aerosol or piezoelectric device is utilized, the volatile material may dispensed intermittently at predetermined intervals. FIG. 15A illustrates a first implementation of the fifth mode 600 of dispensing, wherein the implementation does not include a dwell period. A first period of dispensing a volatile material from a first container or cartridge begins at the beginning of day 1 and continues through day 7. A second period of dispensing the volatile material from a second container or cartridge begins at the beginning of day 8 and continues through day 14, and so forth through day 28. Once four containers or cartridges of volatile material are dispensed at day 28, the containers or cartridges are removed and a new set of containers or cartridges is utilized. The containers or cartridges of volatile material may be contained in separated containers or cartridges or may all be contained in a single container with multiple compartments.

FIG. 15B illustrates a second implementation of the fifth mode 600 of dispensing including a dwell period of, for example, about a day. Referring to FIG. 15B, the first period of dispensing continues through day 7 but the second period of dispensing does not begin until, for example, the beginning of day 9. The second period of dispensing continues through day 15 but the third period of dispensing does not begin until the beginning of day 17, and so forth through the fourth period of dispensing. In practice, the dwell period may be much shorter than a day, perhaps only a few minutes or a few hours, and may be any time period between a second and a day, and preferably any time period between a minute and an hour.

FIG. 15C illustrates a third implementation of the fifth mode 600 of dispensing including an overlap period of, for example, about a day. Referring to FIG. 15C, the first period of dispensing continues through day 7 while the second period of dispensing begins, for example, at the beginning of day 7 and continues through day 13 and the third period of dispensing begins, for example, at the beginning of day 13 and continues through day 19, etc. In practice, the overlap period may be much shorter than a day, perhaps only a few minutes or a few hours, and may be any time period between a second and a day, and preferably any time period between a minute and an hour.

The periods of dispensing each of the fragrances for the fifth mode 600 are preferably between about 4 days and about 10 days, more preferably between about 6 days and about 8 days, and most preferably about 7 days. The periods of dispensing for each of the fragrances need not be the same. For example, the periods of dispensing may be adapted to the volatility and/or other characteristics of each fragrance such that each fragrance has a different period of dispensing within the stated ranges.

Although the various implementations of the fifth mode 600 of dispensing are described as emitting volatile materials from multiple containers, cartridges, or compartments, wherein the volatile materials are the same, different volatile materials may also be utilized. Still further, although the implementations of the fifth mode 600 are described as utilizing four containers, cartridges, or compartments of volatile material, any number of containers, cartridges, or compartments equal to or greater than two may be utilized. For example, the various implementations of the fifth mode 600 of dispensing, as illustrated in FIGS. 15A-C and 16, may alternatively include repeated shortened periods of dispensing of two, three, four, or more different fragrances from separate containers or from separate compartments of a cartridge, wherein the shortened periods of dispensing are tailored to be shorter than or about equal in time to a characteristic time decay period T_{c} as computed for each of the fragrances. The different fragrances could be of a common family of fragrances or could be from different families of fragrances. Thus the fifth mode 600 may be used in addition to any of the first, second, third, and fourth modes 200, 300, 400, 500 described hereinabove to inhibit adaptation and/or habituation to one or more dispensed fragrances.

A first study implementation of the fifth mode 600 of dispensing included 16 women participants (“the respondents”) that were given test units for a three-week in-home use test. The test units included a test box with three outlets and programmable timers corresponding to each of the outlets. Three dispensers capable of each dispensing a single fragrance were connected to the outlets, wherein the dispensers utilized are detailed in Zobele U.S. Pat. No. 6,996,335. The respondents were given their choice of fragrance (from Hawaiian Breeze, Vanilla Passion Fruit, Lavender & Vanilla, Vanilla Breeze, Water Lily Rain, Clean Linen, and Ocean Blue) and each of the dispensers was loaded with such fragrance (all the same for a test unit). During the three-week period, a first of the timers was programmed to emit fragrance from a first of the dispensers for the first week, a second of the timers was programmed to emit fragrance from a second of the dispensers for the second week, and a third of the timers was programmed to emit fragrance from a third of the dispensers for the third week. Emission of each of the fragrances for a week substantially depleted the fragrance from the refill in which it was disposed. During this period, respondents were asked to keep a journal of their experiences and, at the end of the three-week period, completed a questionnaire.

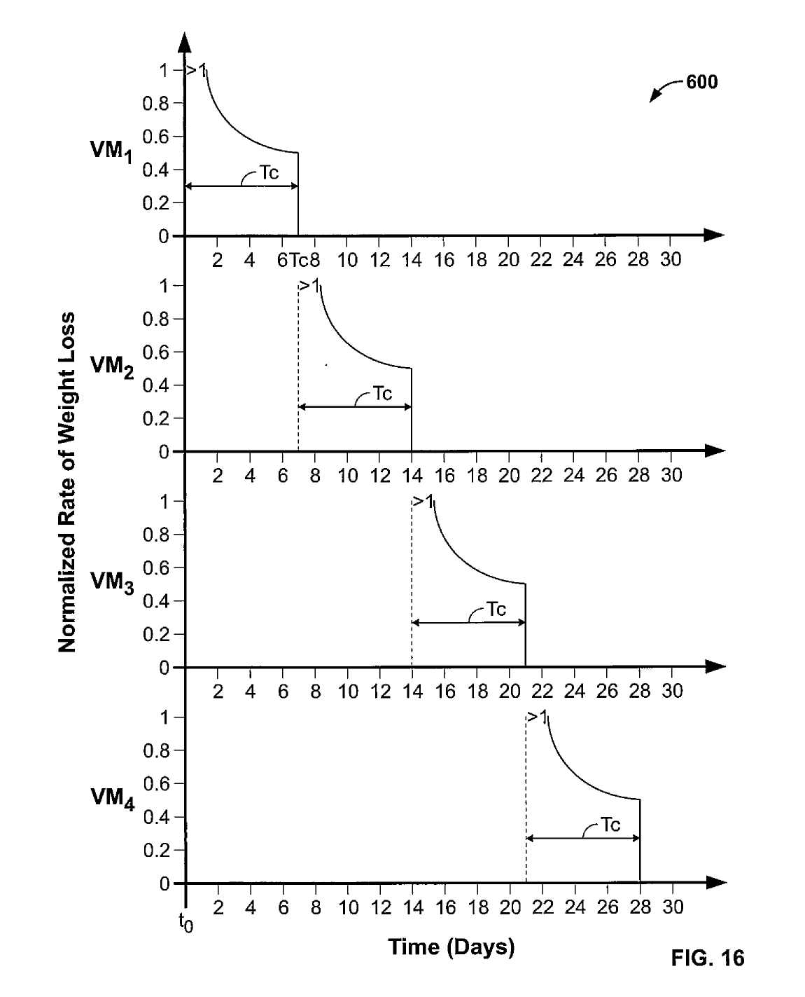

The graph of FIG. 17 plots respondent reactions to both test unit functionality and consumer appeal for the first study implementation. Test unit functionality deals with how well the test unit delivers on the stated core benefit. For test unit functionality, a rating of high means the test unit worked as expected and there were no significant technical problems and few if any usability issues, a rating of moderate means the test unit did not always function consistently or could not be used in all expected situations, and a rating of low means the test unit did not function as expected and there were significant technical challenges to fix. Consumer appeal deals with how interested the respondent was in the benefit the test unit was designed to deliver. For consumer appeal, a rating of high means the respondent saw the benefit as completely new or significantly better than what they experience currently, a rating of moderate means the respondent saw the benefit, but it was polarizing or delivered in a manner that resulted in trade-offs, and a rating of low means the respondent did not see the benefit as important or the benefit was already being met by an existing product.

As can be seen from the graph of FIG. 17, half of the respondents rated both the test unit functionality and the consumer appeal as high. Further, only one respondent rated consumer appeal as low and only one respondent rated test unit functionality as low. One should note that only 14 respondents are included in the graph of FIG. 17 because the responses from the other two respondents were either not recorded or weren't considered reliable.

A second study implementation of the fifth mode 600 of dispensing included 16 women participants (“the respondents”) that were given test units for a three-week in-home use test. The test units, set-up, and directions given to the respondents were identical to those of the first study implementation of the fifth mode 600, except that a different fragrance was used from week to week, wherein each respondent selected a fragrance combination. The fragrance combinations included (in order of week 1, week 2, and week 3): (1) Hawaiian Breeze, Vanilla Passion Fruit, and Hawaiian Breeze, (2) Vanilla Breeze, Pumpkin Pie, and Vanilla Breeze, (3) Clean Linen, Sunny Days, and Clean Linen, and (4) Lavender Meadow, Lavender & Vanilla, and Lavender Meadow.

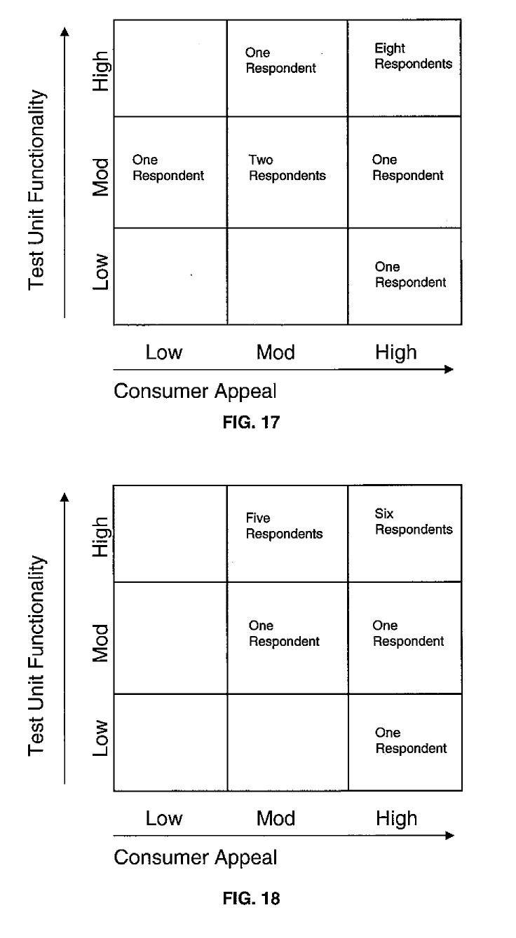

The graph of FIG. 18 plots respondent reactions to both test unit functionality and consumer appeal for the second study implementation, wherein test unit functionality and consumer appeal and ratings therefor are defined above with respect to the first study implementation. As can be seen from the graph of FIG. 18, most of the respondents rated test unit functionality as high and consumer appeal as moderate or high. Further, only one respondent rated test unit functionality as low and no respondent rated consumer appeal as low. Again, one should note that only 14 respondents are included in the graph of FIG. 18 because the responses from the other two respondents were either not recorded or weren't considered reliable.

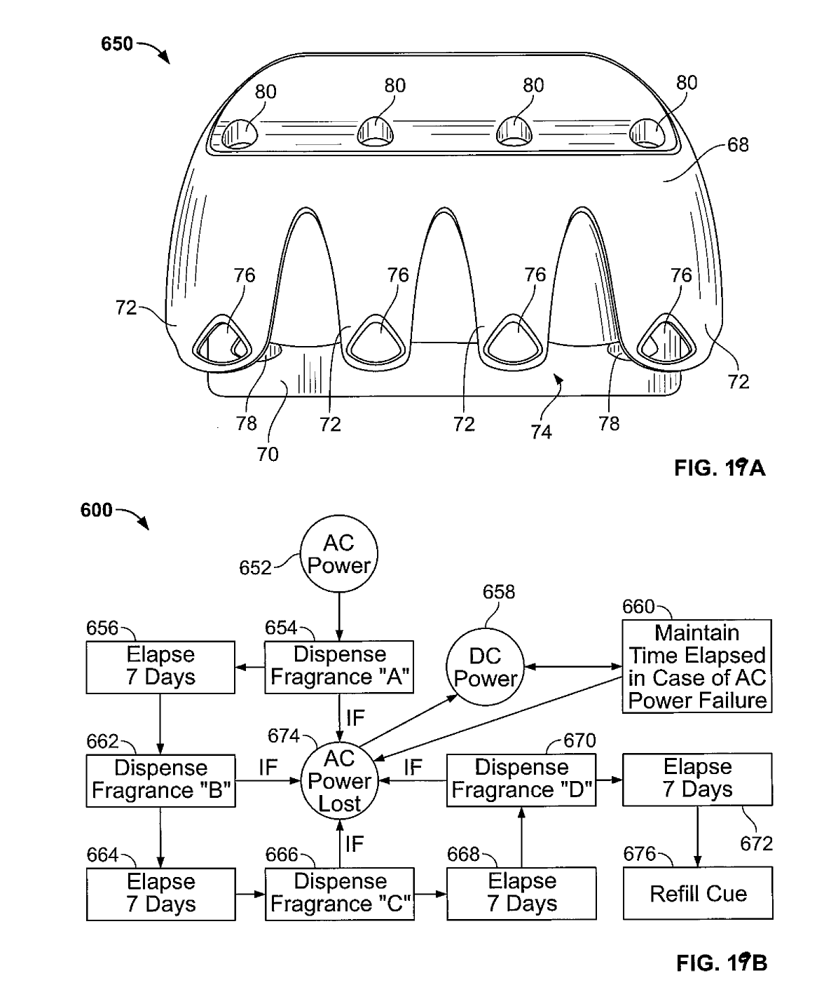

A volatile material dispenser that is similar to the volatile material dispenser 50 discussed with regard to FIGS. 1-3 hereinabove or that is similar to the volatile material dispenser 100 discussed with regard to FIGS. 4-8 hereinabove may be used to execute the various implementations of the fifth mode 600 of dispensing. Referring to FIG. 19A, for example, a volatile material dispenser 650 that accommodates four containers 52 is illustrated and is otherwise identical in structure and operation to the volatile material dispenser 100. It is contemplated that the volatile material dispensers 50, 100, 650 could include non-olfactory cues that indicate levels of fragrances remaining. For example, an LED light or an audio tone could be activated when a level of at least one of the fragrances reaches a minimum threshold.

FIG. 19B illustrates a diagram representing a sample execution of an implementation of the fifth mode 600 of dispensing described hereinabove with regard to FIGS. 15A-15C. At block 652, AC power is provided to a device to execute the fifth mode 600 of dispensing. After AC power is supplied to the device, the device dispenses fragrance “A” at block 654 for a period of, for example 7 days, as represented by block 656. Subsequent to execution of the block 656, fragrance “B” is dispensed at block 662 for a period of, for example 7 days, as represented by block 664. Subsequent to execution of the block 664, fragrance “C” is dispensed at block 666 for a period of, for example 7 days, as represented by block 668, and subsequent to execution of the block 668, at block 670 the device dispenses fragrance “D” for a period of, for example 7 days, as represented by block 672.

A DC power supply represented by block 658, for example, a rechargeable battery pack, provides uninterrupted power to the device at block 660, which represents a mechanism that keeps track of time from a start of the fifth mode 600 of dispensing. In the case of an AC power failure at any time during execution of the fifth mode 600 of dispensing, as indicated by block 674, the fifth mode 600 of dispensing resumes execution at the proper instance when the AC power is restored. Subsequent to the execution of the block 672, a refill cue, for example, an LED light or an audio tone as represented by block 676 may be activated to signal that a fragrance refill is needed.

During any of the burst, odor eliminating, or palate cleansing emission periods as described hereinabove, one or more fragrances may be emitted simultaneously or sequentially. Still further, the one or more fragrances may also be alternated.

Various details shown in FIGS. 1-19B may be modified as will be apparent to those of skill in the art without departing from the disclosed principles. Other methods and materials suitable for executing the modes of dispensing of the present invention may also be utilized.

### INDUSTRIAL APPLICABILITY

Modes of dispensing a volatile material and an apparatus for executing the modes of dispensing while inhibiting adaptation and/or habituation in response to the volatile material have been presented. It has been discovered that a mode of dispensing fragrances having 6 hour emission periods is unexpectedly more effective at inhibiting adaptation and/or habituation to the fragrances than a mode of dispensing similar fragrances having 45 minute emission periods. In addition, cleansing of the environment with bursts of a particular fragrances or a long off or dwell period may also or in addition inhibit adaptation and/or habituation.

Numerous modifications to the present invention will be apparent to those skilled in the art in view of the foregoing description. Accordingly, this description is to be construed as illustrative only and is presented for the purpose of enabling those skilled in the art to make and use the invention and to teach the best mode of carrying out same. The exclusive right to all modifications within the scope of the impending claims is expressly reserved. All patents, patent publications and applications, and other references cited herein are incorporated by reference herein in their entirety.

We claim:

1. A method of dispensing two or more volatile materials, the method comprising the steps of:
providing a volatile material dispenser having at least one diffusion element and adapted to accommodate two or more containers having volatile materials therein, wherein each of the volatile materials is subjected to a diffusion element;
emitting a first of the volatile materials at a first intensity level for a first period of time between about 30 minutes and about 2 hours; and
emitting a second of the volatile materials at a second intensity level for a second period of time following the first period of time, wherein the second period of time is between about 1 minute and about 30 minutes and the second intensity level is greater than the first intensity level; and
repeating the step of emitting the first volatile material.

2. The method of claim 1, wherein the first period of time is between about 45 minutes and about 1 hour and the second period of time is between about 5 minutes and about 20 minutes.

3. The method of claim 1, further including the step of:
emitting the second volatile material at the second intensity level during the first period of time.

4. The method of claim 1, wherein the volatile materials are fragrances and the first and second volatile materials are different.

5. The method of claim 1, further including the steps of:
emitting a third volatile material at the first intensity level for a third period of time following the second period of time, wherein the third period of time is between about 30 minutes and about 2 hours; and
emitting the second volatile material at the second intensity level for the second period of time immediately following the third period of time.

6. The method of claim 5, wherein the first and third periods of time are between about 45 minutes and 1 hour and the second period of time is between about 5 minutes and about 20 minutes.

7. The method of claim 5, further including the step of continuously repeating the pattern of emission of the first volatile material, the second volatile material, the third volatile material, and the second volatile material.

8. A method of dispensing two or more volatile materials, the method comprising the steps of:
providing a volatile material dispenser having two or more diffusion elements and adapted to accommodate two or more containers having volatile materials therein, wherein each of the volatile materials is subjected to one of the diffusion elements;
emitting a first of the volatile materials for a first period of time;
emitting a second of the volatile materials for a second period of time following the first period of time; and
refraining from emission of any volatile material for a third period of time following the second period of time;
wherein the first, second, and third periods of time make up an emission cycle that is repeated and wherein the emission cycle is greater than or equal to about 24 hours.

9. The method of claim 8, wherein each of the diffusion elements includes a heating element.

10. The method of claim 8, wherein the emission cycle is between about 24 hours and about 7 days.

11. The method of claim 10, wherein the emission cycle is about 24.

12. The method of claim 11, wherein the second period of time begins immediately following the first period of time, the third period of time begins immediately following the second period of time, each of the first and second periods of time is between about 3 hours and about 12 hours, and the third period of time is between about 1 hour and about 5 hours.

13. The method of claim 12, wherein each of the first and second periods of time is between about 5 hours and about 10 hours and the third period of time is between about 3 hours and about 4 hours.

14. The method of claim 13, wherein each of the first and second periods of time is about 10 hours and the third period of time is about 4 hours.

15. The method of claim 12, wherein the first and second time periods are different.

16. The method of claim 9, wherein each of the first and second periods of time make up between about 10% and about 50% of the emission cycle and the third period of time makes up between about 1% and about 30% of the emission cycle.

17. The method of claim 16, wherein each of the first and second periods of time make up between about 20% and about 40% of the emission cycle and the third period of time makes up between about 10% and about 20% of the emission cycle.

18. The method of claim 8, further including the step of:
emitting a third volatile material for a fourth period of time following the second period of time and before the third period of time;
wherein the first, second, third, and fourth periods of time make up the emission cycle that is repeated.

19. The method of claim 18, wherein the emission cycle is between about 24 hours and about 7 days.

20. The method of claim 19, wherein the second period of time begins immediately following the first period of time, the fourth period of time begins immediately following the second period of time, the third period of time begins immediately following the fourth period of time, each of the first, second, and fourth periods of time is between about 3 hours and about 12 hours, and the third period of time is between about 1 hour and about 5 hours.

21. The method of claim 20, wherein each of the first, second, and fourth periods of time is between about 5 hours and about 10 hours and the third period of time is between about 3 hours and about 4 hours.

22. The method of claim 21, wherein the first, second, and fourth periods of time are about 7 hours and the third period of time is about 3 hours.

23. The method of claim 18, wherein each of the first, second, and fourth periods of time make up between about 10% and about 50% of the emission cycle and the third period of time makes up between about 1% and about 30% of the emission cycle.

24. The method of claim 23, wherein each of the first, second, and fourth periods of time make up between about 50% and about 50% of the emission cycle and the third period of time makes up between about 10% and about 20% of the emission cycle.

25. The method of claim 24, further including the step of creating an overall greater volatile material strength over time than would be created with an alternation of volatile materials wherein such volatile materials are emitted for about 45 minutes.

26. The method of claim 8, further including the step of:
randomly determining at least one of the first, second, and third periods of time.

27. A method of dispensing two or more volatile materials, the method comprising the steps of:
providing a volatile material dispenser having at least one diffusion element and adapted to accommodate two or more containers having volatile materials therein, wherein each of the volatile materials is subjected to a diffusion element;
emitting a first volatile material from a first of the containers for a first generally continuous period of time, wherein after the first period of time, the first volatile material in the first container is substantially depleted; and
emitting a second volatile material from a second of the containers for a second generally continuous period of time, wherein after the second period of time, the second volatile material in the second container is substantially depleted.

28. The method of claim 27, further including the step of:
emitting a third volatile material from a third containers for a third generally continuous period of time, wherein after the third period of time, the third volatile material in the third container is substantially depleted.

29. The method of claim 28, wherein each of the first, second, and third periods of time is between about 4 days and about 10 days.

30. The method of claim 29, wherein the first, second, and third periods of time are each about 10 days.

31. The method of claim 29, wherein each of the first, second, and third periods of time begins immediately following a previous period and, thus, do not overlap or include a gap therebetween.

32. The method of claim 27, further including the step of:
emitting a fourth volatile material from a fourth of the containers for a fourth generally continuous period of time that follows the third periods of time, wherein after the fourth period of time, the fourth volatile material in the fourth container is substantially depleted.

33. The method of claim 32, wherein each of the first, second, third, and fourth periods of time is between about 4 days and about 10 days.

34. The method of claim 33, wherein each of the first, second, third, and fourth periods of time is between about 6 days and about 8 days.

35. The method of claim 34, wherein each of the first, second, third, and fourth periods of time is about 7 days.

36. The method of claim 33, wherein the first, second, third, and fourth periods of time are not all the same.

37. The method of claim 35, wherein the first, second, third, and fourth periods of time begin immediately following a previous period, and thus, do not overlap or include a gap therebetween.
